(19) United States
(12) Patent Application Publication
(10) Pub. No.: US 2003/0130610 A1
(43) Pub. Date: Jul. 10, 2003

(54) Aortic balloon catheter with improved positioning and balloon stability

(76) Inventors: Larry F. Mager, Pleasanton, CA (US); Jerome B. Riebman, Sunnyvale, CA (US); Raymond Bertolero, Danville, CA (US); Arthur A. Bertolero, Danville, CA (US)

Correspondence Address: GREGORY SMITH & ASSOCIATES, 3900 NEWPARK MALL ROAD, 3RD FLOOR, NEWARK, CA, 94560, US

(21) Appl. No.: 10/297,858

(22) PCT Filed: Feb. 2, 2001
(86) PCT No.: PCT/US01/03395

(51) Int. Cl. A61M 25/10; A61M 25/14; A61M 5/14

(52) U.S. Cl. 604/6.16; 604/7

(57) ABSTRACT

A multi-lumen aortic balloon catheter is disclosed. The catheter is designed to assist surgeons in more effectively performing cardiovascular surgery, particularly cardiopulmonary bypass (CPB) surgery. In one aspect the catheter is inserted into a femoral artery and threaded through the artery to the aortic arch where it is positioned so that the balloon is positioned in the ascending aorta. When inflated, the balloon (preferably a cylindrical design) blocks the aortic arch between the great arteries and the coronary ostia. A cardioplegia solution is delivered to the heart through an internal lumen in the catheter to slow the heart. Blood from a cardiopulmonary machine is transported through a blood flow lumen of the catheter to be delivered antegrade flow throughout the arteries. The catheter has a distal portion having fewer lumens than are present in a proximal portion. An alternative multilumen aortic balloon catheter is disclosed that is inserted through a patient's aorta.

### CROSS REFERENCE

This application claims priority to U.S. patent application filed Feb. 4, 2000, as Ser. No. 60/180,233 and is a continuation-in-part thereof.

### INTRODUCTION

### Technical Field

This invention relates to an improved multi-lumen balloon catheter for blocking the ascending aorta and delivering blood in a heart-surgery patient.

### Background

To better understand the background and problems faced by those of skill in this area of technology it is useful to understand the basic workings of the heart and circulatory system. The following discussion refers to schematics of the heart shown in FIGS. 1 and 2. The human heart is a muscular pump having four separate cavities and a series of valves allowing blood to pass in one direction only. Mammals, including humans, have a double circulatory system. Blood that has released oxygen to the tissues (9 and 14) and has absorbed carbon dioxide from them (venous blood) is returned to the heart through the superior and the inferior venae cavae (11 and 10). This blood enters the right auricle (3), whose contractions cause the blood to pass through the tricuspid valve (16) in the right ventricle (1). The contractions of the right ventricle pass the blood through the pulmonary semilunar valves (17) and along the two pulmonary arteries (5) into the lungs (6). In the lungs, the blood is oxygenated and returns to the heart through the pulmonary veins (7) and thus enters the left auricle (4). This chamber contracts and passes the blood through the bicuspid, or mitral, valve (15) into the left ventricle (2), whose contractions force the blood through the aortic semilunar valve (18) into the aorta (12 and 13), which is the biggest artery of the body and to other parts of the body through, i.e., the great arteries 8. Thus the right side of the heart serves mainly to pump deoxygenated blood through the lungs, while the left side pumps oxygenated blood throughout the rest of the body. This is represented as a flow schematic in FIG. 2, where similar numbers refer to similar parts of the heart. The heart varies the output by varying the volume of blood admitted into the ventricles each time the latter are filled and also by varying the rate of contraction (faster or slower heartbeat). The left side of the heart (left auricle and ventricle) has to circulate the blood through all parts of the body, except the lungs, and has thicker and more strongly muscular walls than the right side, which has to perform the pulmonary blood circulation only. For proper functioning, the left side and the right side must be accurately interadjusted, both with regard to the contraction rate of the respective chambers and with regard to the output of blood. When functional disorders of the heart occur, it may be necessary to examine the heart to determine the problem and possibly perform surgery or provide treatment.

In performing examinations or treatments of a subject's heart, or performing surgery on the heart, it is often necessary to reduce the rate at which it normally beats or stop its beating completely. This allows a physician to observe, or operate on, the heart more easily. However, by reducing or stopping the heart rate (i.e., cardioplegia), blood will not be adequately circulated to the rest of the body. Thus, it is generally necessary to circulate the blood using some type of extracorporeal blood circulating means that regularly circulates oxygen-rich blood through the arteries, collects oxygen-depleted blood returning through the veins, enriches the oxygen-depleted blood with additional oxygen, then again circulates the oxygen-rich blood.

The types of examinations, treatments and operations that require some degree of cardioplegia or drug delivery and extracorporeal blood circulation include open heart surgery and less-invasive heart surgery to perform single or multiple coronary artery bypass operations, correct malfunctioning valves, etc. Others include, but are not limited to, myocardial revascularization, balloon angioplasty, correction of congenital defects, surgery of the thoracic aorta and great vessels, and neurosurgical procedures.

The extracorporeal blood circulation generally requires the use of some type of heart-lung machine, i.e., a cardiopulmonary machine. This has the threefold function of keeping the replacement blood in circulation by means of a pumping system, of enriching with fresh oxygen the blood of low oxygen content coming from the patient's body, and regulation of patient temperature. The system shown in FIG. 3 diagrammatically describes the manner in which such a machine works.

The venous blood, before it enters the right auricle of the heart is diverted into plastic tubes (20), generally by gravity flow. The tubes are positioned to receive the blood from the superior and inferior venae cavae (shown as 11 and 10 in FIG. 1). This blood, which has circulated through the body and consequently has a low oxygen content is collected in a reservoir (21). A blood pump (22) is used to pump the blood through a heat exchanger (23) and artificial lung (24). The heat exchanger (23) and artificial lung (24) may be one of several designs to regulate blood temperature and increase the oxygen content of the blood. Modern designs use advanced membrane technology to achieve the oxygenation, which is similar to the way red blood cells absorb oxygen from the human lung. The oxygenated blood then passes through a filter (25) and is returned to the patient. Losses of blood occurring during the course of the operation are compensated by an additional blood reservoir (26). Collected blood is passed through a defoamer (27) and is likewise passed to the reservoir 21, heat exchanger (23) and artificial lung (24). Before starting the cardiopulmonary bypass machine the extracorporeal circuit is filled with one or two liters of saline solution. In circulating the oxygenated blood to the body from filter 25, it can be pumped through a line 28 by attaching the line to a catheter leading into the aorta or one of its major branches and pumping the blood through the catheter. However, when the heart is to be operated on, it must be free of blood and sometimes the heart beat must be reduced or stopped completely. Referring again to FIG. 1, blood is prevented from entering the heart by blocking the ascending aorta 12 near the semilunar valve 18 while at the same time preventing blood from entering the right auricle 3 by withdrawing blood through the superior vena cavae 11 and inferior vena cavae and 10. Blocking the ascending aorta may be achieved by clamping or preferably by balloon blockage. At the same time that blood is prevented from flowing through the heart, a cardioplegia solution is administered locally to the heart to arrest the heart. Thus, there is a need for a device that allows a heart specialist to locally administer cardioplegia to the heart, block the flow of blood to the heart, while at the same time circulating oxygenated blood to the patient's body, particularly through the great arteries (8 in FIG. 1), to ensure all limbs and tissues remain undamaged during the heart examination or operation. Several devices are described in the literature to address the need for an appropriate device. One example is disclosed in U.S. Pat. No. 5,312,344 issued May 17, 1994 to Grinfeld, et al.

Another example can be seen in U.S. Pat. No. 5,433,700 issued Jul. 18, 1995 to Peters. This patent describes a process for inducing cardioplegic arrest of a heart which comprises maintaining the patient's systemic circulation by peripheral cardiopulmonary bypass, occluding the ascending aorta through a percutaneously placed arterial balloon catheter, venting the left side of the heart, and introducing a cardioplegia agent into the coronary circulation. As part of the disclosure a multichannel catheter is disclosed which provides channels for the cardioplegia solution, a fluid transportation to inflate the balloon and a lumina for instrumentation. Further patents in this family include U.S. Pat. No. 5,725,496 and U.S. Pat. No. 5,971,973.

Another example of a device is found in U.S. Pat. No. 5,478,309 issued Dec. 26, 1995 to Sweezer, et al. This is a rather complex device and system of venous perfusion and arterial perfusion catheters for use in obtaining total cardiopulmonary bypass support and isolation of the heart during the performance of heart surgery. One of the multichannel catheters described in the patent for delivering cardioplegia solution to the heart while blocking the ascending aorta and circulating perfused blood.

Another device is described in U.S. Pat. No. 5,458,574 issued Oct. 17, 1995 to Machold, et al. It shows a multichannel catheter which has channels for fluid to blow up balloons for blocking the aorta, a channel for cardioplegia solution and a channel for instruments for examining the heart.

Still another patent, U.S. Pat. No. 5,452,733 issued Sep. 26, 1995 to Sterman, et al.

Still another patent application filed as PCT/US 94/09938 having international publication No. WO95/08364 filed Sep. 1, 1994 in the name of Evard, et al. describes an endovascular system for arresting the heart. PCT International Application number PCT/US No. 94/12986 published as Publication No. WO95/15192, filed Nov. 10, 1994 in the name of Stevens, et al. provides a description of a partitioning device that is coupled to an arterial bypass cannula. The description provides for the cannula to be introduced to the femoral artery where the partitioning device has a balloon at the end of the flexible tube to block the ascending aortic artery and allow blood to circulate through a lumen.

Another patent, U.S. Pat. No. 5,584,803, issued Dec. 17, 1996 to Stevens, et al. describes an endovascular device for partitioning a patient's ascending aorta with a balloon catheter. Additional patents claiming priority to the '803 patent have also issued.

Another patent is U.S. Pat. No. 5,868,703, which discloses a unique multichannel catheter.

While each of these documents describe a step of progress in the art, the devices disclosed have certain shortcomings that can be improved upon. For example, some of the designs of the balloon catheters can result in kinking of the line as it transcends the aortic arch to position the balloon. All of the designs use a catheter that is of the same cross-sectional diameter for the length of the catheter. Some of the references suggest shaping (i.e., prebending) the distal end of the catheter on the theory that the shaping or precurving the catheter will aid in getting the tip to more easily transcend the aortic arch. This requires, however, that a straightening guide wire be used to keep the shaped distal end straight as it is pushed along the femoral artery towards the aortic arch. The wire is withdrawn as it reaches the aortic arch to allow the shaped distal end to go around the arch. It has been discovered, however, that such shaping can have an adverse effect on positioning the balloon in the aortic arch—instead of centering the balloon, it tends to position off center and not properly block the arch.

It has now been discovered that by narrowing the distal portion of a balloon catheter and maintaining the distal portion straight, while at the same time reducing the “kinkability” and increasing the flexibility, the balloon can be more effectively positioned. Also, by employing a balloon having an elongated design, the positioning is improved and the risk of trauma is reduced.

### SUMMARY

One aspect of this invention is a balloon catheter for delivering blood to an animal while blocking the aortic arch between the great arteries and the coronary ostia. The balloon catheter has a distal portion conjoined with a proximal portion. The distal portion comprises:

(a) an elongated, flexible shaft having distal and proximal ends and further having at least two lumens extending about the length of the shaft independent of and parallel to each other,

(b) the first lumen having an opening at both the distal and proximal ends of the shaft,

(c) an inflatable balloon integrated into the shaft near the distal end of the shaft,

(d) the second lumen having an opening at the proximal end of the shaft and an opening in fluid communication with the interior of the inflatable balloon, and

(e) the shaft having a non-traumatic distal tip and a length sufficient to traverse the aortic arch of a human.

The proximal portion comprises a multi-lumen blood delivery portion having distal and proximal ends and being conjoined with the proximal end of the shaft at the distal end of the multi-lumen blood delivery portion. The multi-lumen blood delivery portion further comprises:

(a) a first lumen defined by a surrounding wall extending the length of the multi-lumen portion and being closed at its distal end but open at its proximal end for receiving extracorporeal blood from a cardiopulmonary machine,

(b) a second lumen (i) extending the length of the multi-lumen portion parallel to the first lumen but independent thereof and (ii) open at its distal end, and

(c) third lumen that (i) is independent of and parallel to the first and second lumens, (ii) extends the length of the three-lumen portion, and (iii) is open at the distal end of the third lumen, wherein a plurality of outlet ports extend along the wall at the distal region of the proximal portion, the ports being in fluid communication solely with the interior of the first lumen.

The proximal end of the distal portion is conjoined with the distal end of the proximal portion so that the first lumen of the distal portion is in fluid communication solely with the second lumen of the proximal portion and the second lumen of the distal portion is in fluid communication solely with the third lumen of the proximal portion.

Another aspect of this invention is a method of performing cardiovascular surgery on a patient having a need thereof, which method comprises:

(A) inserting the balloon catheter as described herein into the patient through the patient's femoral artery so that the balloon is positioned in the ascending aorta between the patient's coronary ostia and great arteries;

(B) expanding the balloon to substantially block fluid communication between the patient's heart and the aorta;

(C) providing cardioplegia through the balloon catheter to the patient's heart to slow the heart rate;

(D) circulating blood from a cardiopulmonary machine through the balloon catheter to the patient's aorta and connected arteries; and

(E) performing the cardiovascular surgery on the patient.

Another aspect of this invention is a method for preparing a balloon catheter of this invention. The method comprises:

(A) preparing a distal portion of the catheter that comprises:

(1) an elongated, flexible shaft having distal and proximal ends and further having at least two lumens extending about the length of the shaft independent of and parallel to each other,

(2) the first lumen having an opening at both the distal and proximal ends of the shaft,

(3) an inflatable balloon integrated into the shaft near the distal end of the shaft,

(4) the second lumen having an opening at the proximal end of the shaft and an opening in fluid communication with the interior of the inflatable balloon, and

(5) the shaft having a non-traumatic distal tip and a length sufficient to traverse the aortic arch of a human;

(B) preparing a proximal portion of the catheter that comprises a multi-lumen blood delivery portion having distal and proximal ends and being suitable for conjoining with the proximal end of the shaft of (A) at the distal end of the multilumen blood delivery portion. One which multi-lumen blood delivery portion further comprises

(1) a first lumen defined by a surrounding wall extending the length of the multi-lumen portion and being closed at its distal end but open at its proximal end for receiving extracorporeal blood from a cardiopulmonary machine,

(2) a second lumen (i) extending the length of the multi-lumen portion parallel to the first lumen but independent thereof and (ii) open at its distal end, and

(3) third lumen that (i) is independent of and parallel to the first and second lumens, (ii) extends the length of the three-lumen portion and (iii) is open at the distal end of the third lumen, wherein a plurality of outlet ports extend along the wall of the first lumen at the distal portion of the proximal portion, the ports in fluid communication solely with the interior of the first lumen; and

(C) aligning the proximal end of the distal portion with the distal end of the proximal portion so that the first lumen of the distal portion aligns with the second lumen of the proximal portion and the second lumen of the distal portion aligns with the third lumen of the proximal portion; and

(D) permanently conjoining the distal and proximal portions together so that the lumens aligned in part (C) above are in fluid communication with the other.

Another aspect of this invention is a multi-lumen balloon catheter for attachment to a another multi-lumen blood delivery catheter. The first multi-lumen balloon catheter comprises:

an elongated, flexible shaft having distal and proximal ends and further having at least two lumens extending about the length of the shaft independent of and parallel to each other,

the first lumen having an opening at both the distal and proximal ends of the shaft,

an inflatable balloon integrated into the shaft near the distal end of the shaft,

a second lumen having an opening at the proximal end of the shaft and an opening in fluid communication with the interior of the inflatable balloon,

the distal tip of the shaft having a blunt, nontraumatic design, and

the shaft having a length sufficient to traverse the aortic arch of a human.

Still another aspect of this invention is multi-lumen blood delivery catheter having distal and proximal ends and being suitable for conjoining with multi-lumen shaft at the distal end of the first multi-lumen catheter, wherein the other multi-lumen shaft has at least one less lumen than the first multi-lumen catheter. The multi-lumen blood delivery catheter comprises:

(a) a first lumen defined by a surrounding wall extending the length of the multi-lumen catheter and being closed at its distal end but open at its proximal end for receiving extracorporeal blood from a cardiopulmonary machine,

(b) a second lumen (i) extending the length of the multi-lumen catheter parallel to the first lumen but independent thereof and (ii) open at its distal end, and

(c) third lumen that (i) is independent of and parallel to the first and second lumens, (ii) extends the length of the multi-lumen catheter and (iii) is open at its distal end,

wherein a plurality of outlet ports extend along the wall at the distal portion of the three-lumen catheter, the ports in fluid communication solely with the interior of the first lumen.

Another aspect of this invention is a balloon catheter for delivering blood to an animal while blocking the aortic arch between the great arteries and the coronary ostia. The balloon catheter is designed for insertion through the base of a patient's aortic arch. The catheter comprises a distal blood delivery section and proximal blood transport section. The proximal blood transport section has distal and proximal ends and is conjoined with the proximal end of the distal blood delivery section at the distal end of the blood transport section. The blood transport section further comprises (a) a first blood transport lumen defined by a surrounding wall extending the length of the blood transport section open at its proximal end for receiving extracorporeal blood from a cardiopulmonary machine and being open at its distal end, (b) a second lumen (i) extending the length of the blood transport section parallel to the first lumen but independent thereof and (ii) open at its distal end for delivering cardioplegia solution to the heart near the aortic root, and (c) third lumen that (i) is independent of and parallel to the first and second lumens, (ii) extends the length of the three-lumen portion, (iii) is open at its distal end, and (iv) communicates with the interior of an inflatable balloon integrated into the distal region of the blood transport section. The distal blood delivery section comprises an extension of the first lumen of the blood transport section, the extension (i) being of a length to traverse at least a portion of the aortic arch, (ii) being in fluid communication with the first blood transport lumen, an (iii) having a plurality of outlet ports for delivery of blood in an antegrade fashion to the aorta. The proximal end of the distal blood delivery section is conjoined with the distal end of the proximal blood transport section so that the extension of the first lumen is in fluid communication solely with the blood transport lumen of the proximal portion.

Still another aspect of the invention is a method of performing cardiovascular surgery on a patient having a need thereof using the balloon catheter for aortic insertion. The method comprises (A) inserting the balloon catheter as described immediately above into the patient through the patient's aortic artery to position the balloon catheter so that the balloon is positioned in the ascending aorta between the patient's coronary ostia and great arteries and the blood delivery section is positioned to traverse a portion of the patient's aortic arch; (B) inflating the balloon with a fluid transported through the third-lumen to substantially block fluid communication between the patient's heart and the aorta; (C) providing cardioplegia through the second lumen of the blood transport section to the patient's heart to slow the heart rate; (D) circulating blood from a cardiopulmonary machine through the outlet ports of the blood delivery section of the first lumen to the patient's aorta and connected arteries; and (E) performing the cardiovascular surgery on the patient.

Other aspects of the invention may be apparent to one of skill in the art upon reading the full specification and claims presented herein.

### DESCRIPTION OF THE DRAWINGS

In the accompanying drawings:

FIG. 1 is a diagram of a mammal's heart and circulatory system showing the approximate configuration of the heart.

FIG. 2 is a schematic representative of how a mammalian heart works without regard to its configuration.

FIG. 3 is a schematic representation of how a cardiopulmonary machine works with a heart.

FIG. 4 is a longitudinal cross-section view of the proximal portion of the balloon catheter of this invention showing the interrelationship between the major parts of the proximal portion.

FIG. 5A is a perpendicular cross-section taken along lines 5--5 of 4.

FIG. 5B shows a closely related configuration taken along line 5--5 of FIG. 4.

FIG. 5C shows a slight modification of the cross-section taken along the line of 5--5 of FIG. 4.

FIG. 5D shows a cross-section analogous to that of 5B, but where the proximal portion of the catheter of the invention has 4 lumens instead of 3.

FIG. 5E shows a cross-section analagous to that of 5B, but where the proximal portion of the catheter of the invention has 3 lumens with the two smaller lumens positioned adjacent instead of 180° from each other as shown in 5A or 5B.

FIG. 6A shows a cross-section of the longitudinal axis of a slightly different configuration of the proximal portion of the catheter of this invention.

FIG. 6B shows a cross-section perspective of FIG. 6A.

FIG. 7 shows a perpendicular cross-section taken along lines 5--5 of FIG. 4 and shows the size relationships between the various parts of the multi-channel catheter of this invention.

FIG. 8 shows a cardiopulmonary system using the catheter of this invention.

FIG. 9 is a representation of a preferred aspect of the balloon catheter of the invention having an internal obturator.

FIG. 10A shows a preferred aspect of the balloon catheter of the invention having an internal obturator.

FIG. 10B shows a close up, cross-section view of a portion of 10A.

FIG. 11 shows a partial view of the balloon catheter of the invention having positioning indicators located along the proximal portion of the device.

FIG. 12A shows a full length view of the obturator useful in this invention

FIG. 12B shows a perpendicular cross-section taken along lines J--J of the full length obturator.

FIG. 13 is a schematic representation of how the catheter of the invention works in a mammal's heart and circulatory system.

FIGS. 14A, 14B and 14C show cross-sectional views of the distal portion of the balloon catheter of this invention.

FIGS. 15A, 15B and 15C show how the distal portion transcends the aortic arch.

FIG. 16 shows the balloon catheter of this invention properly positioned within the ascending aortic arch.

FIG. 17 shows an alternative view of a catheter that is inserted through the ascending aorta.

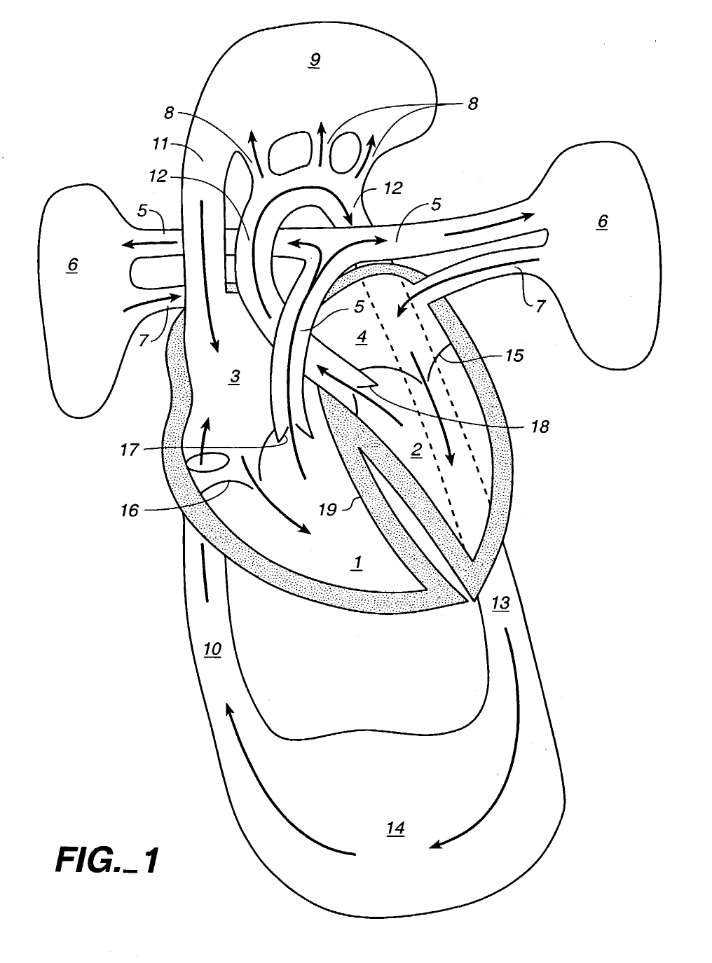

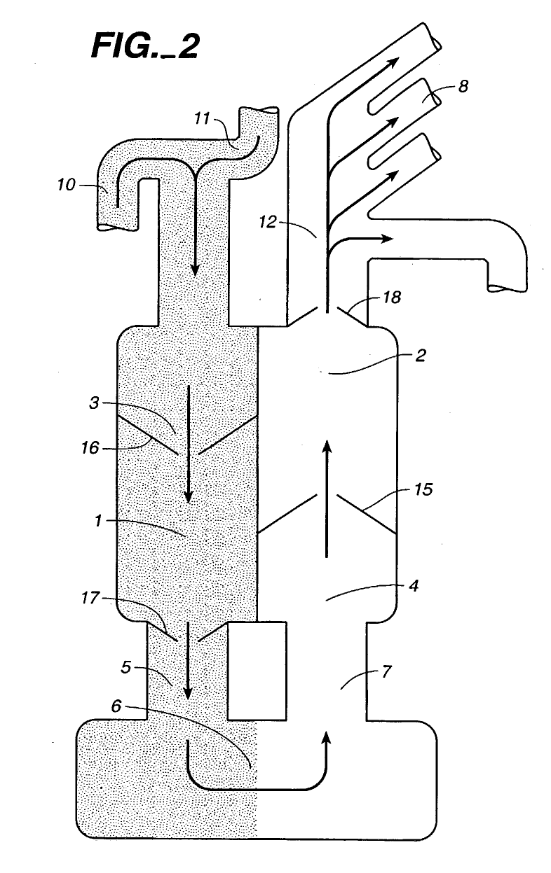

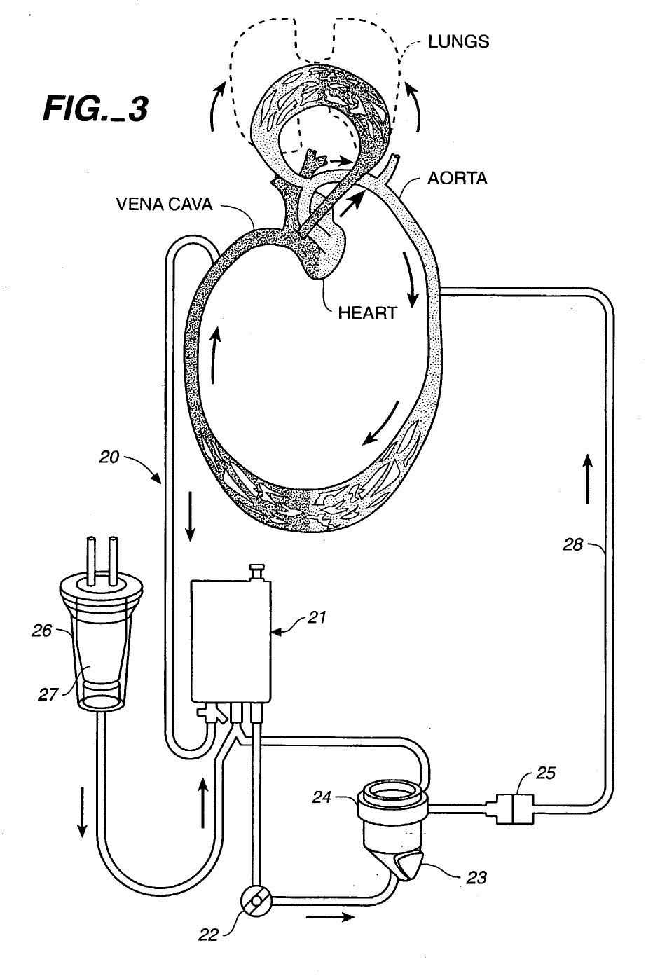

### DESCRIPTION OF SPECIFIC EMBODIMENTS

Overview

One aspect of this invention is a multi-lumen aortic balloon catheter having improved balloon positioning and stability characteristics. It can also be referred to as a remote access perfusion cannula. The catheter is designed to assist surgeons in more effectively performing cardiovascular surgery, whether open-heart surgery or less invasive surgery. It is particularly valuable for cardiopulmonary bypass (CPB) surgery. The catheter performs several functions. It is inserted into a femoral artery and threaded through the artery to the aortic arch where it is positioned so that the balloon is positioned in the ascending aorta. When the balloon is inflated, with saline for example, it blocks the aortic arch between the great arteries and the coronary ostia, thus blocking the flow of blood from the heart. A preferred cylindrical design of the balloon provides very effective aortic occlusion. A cardioplegia solution is delivered from an external source to the heart through an internal lumen in the catheter. This solution will slow or stop the heart. Blood from a cardiopulmonary machine is transported through a blood flow lumen to be delivered antegrade flow throughout the arteries then returned to the cardiopulmonary machine from the inferior and superior vena cavae. The inflated balloon also prevents the blood originating from the cardiopulmonary machine from reaching the heart. The improved balloon positioning and stability characteristics are achieved by designing the catheter to have a distinct distal portion having fewer lumens than are present in a distinct proximal portion in which one of the lumens is a blood carrying lumen that does not extend into the distal portion. Because the distal portion has less space devoted to lumens it is less likely to kink and its flexibility can be better controlled to more easily traverse the aortic arch. By using a balloon design that ensures a greater longitudinal length of the inflated balloon than simply a globular design, the balloon is positioned more firmly with less stress to the ascending aorta tissues.

Another aspect of this invention is a multi-lumen aortic balloon catheter that is inserted through a patient's aorta. Again, the balloon is inflated between the ostia and great arteries to block flow of blood to and from the heart. Cardioplegia is delivered to the heart and blood from a cardiopulmonary machine is delivered antegrade to the aorta and connecting arteries.

In the following discussion, the proximal portion and the distal portion will be discussed separately, then the combination of the two will be discussed. As the catheter is designed to be used in combination with a cardiopulmonary machine (CPM), the terms “distal” and “proximal” refer to the position relative to the CPM. Distal is further away and proximal is closer.

The Proximal Portion

In general, the proximal portion of the multi-lumen balloon catheter of this invention comprises at least one more lumen than the number of lumens in the distal portion (discussed hereinafter). Preferably this will be 3 passageways, with a large, central passageway to maximize the flow of oxygenated blood from a cardiopulmonary machine. It is important to maximize the flow of blood through the large channel while minimizing the outside diameter of the catheter and thus provide adequate systemic extracorporeal blood flow for the vast majority of patients in which the catheter is used. Of the available passage space in the catheter of this invention, a majority, and preferably at least about 70% is allocated to this large passageway to maximize the flow. Preferably about 80% and more preferably about 90% of the available passageway volume, is used for the flow of perfused blood to the arterial side of a patient in need of supplementary, extracorporeal blood circulation. The other channels, at least two, comprise the remainder of the available volume (i.e., less than 50%, but generally about 10%-30%) with each channel preferably integrated into the wall of the large central passageway. Generally, the available volume is determined by calculating the area of a cross-section of each longitudinal passageway and multiplying by the length. Since the length is about the same in each case, the relative volume for each channel will be directly proportional to the cross-sectional area of each passageway.

More specifically, proximal portion of the multi-lumen catheter has distal and proximal regions and comprises a large central, first channel, i.e., a passageway or lumen. This channel extends substantially the length of the proximal portion of the catheter, is closed at its distal end, but has a plurality of certain outflow openings for extracorporeal blood flow along its length towards the proximal region, as discussed in greater detail hereinafter. The catheter has at least second and third channels, each of which extends the length of the catheter, parallel to the first channel but independent thereof. These additional channels are preferably integrated into the wall of the first channel. The multi-lumen catheter further has a plurality of openings near the distal end of said catheter communicating with the first large channel. The openings are said to be “upstream” of the distal end of the proximal portion. In general the length of the proximal portion will be less than 100 cm, preferably about 60-90 cm.

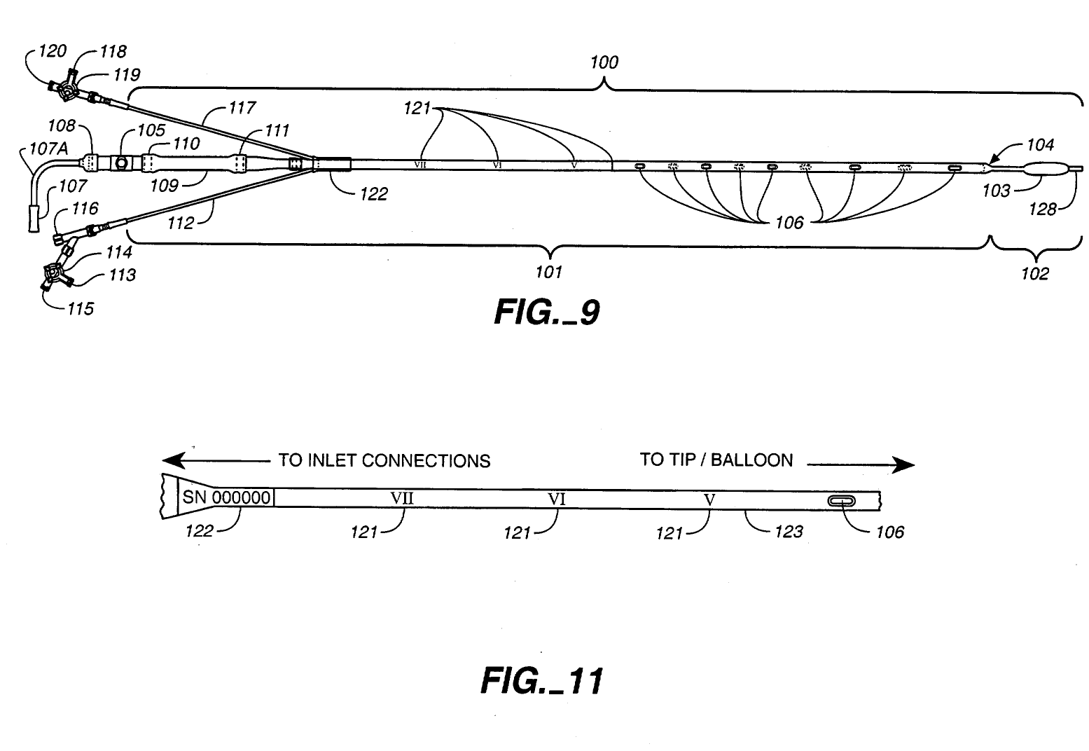

In addition, the multi-lumen catheter of the invention preferably comprises an obturator, i.e., a shaft that snugly fits into the length of the large, blood-carrying channel of the catheter. The obturator may be viewed as a flexible shaft for slidingly and snugly fitting into the first large lumen for blood delivery. The cross-sectional design corresponds to the cross-sectional design of the blood-delivery lumen. Preferably the obturator is beveled at its distal end. Various aspects of the obturator are shown in FIGS. 9-13, and are discussed in more detail hereinafter.

The Distal Portion

The distal portion of the catheter of this invention in its relaxed condition (i.e., prior to insertion into an aorta and positioning to conform to the aortic arch) is preferably straight, that is, it is not shaped or preshaped in an attempt to have it conform to or approximate the aortic arch in some way. In practice, it has been found that “pre-bent” or preshaped catheters do not always orient the occlusion balloon in a position that allows the balloon to expand in a manner that ensures the balloon forces are perpendicular to the wall of the ascending aorta. This can then result in balloon instability due to the compliant nature of aortic occlusion balloons.

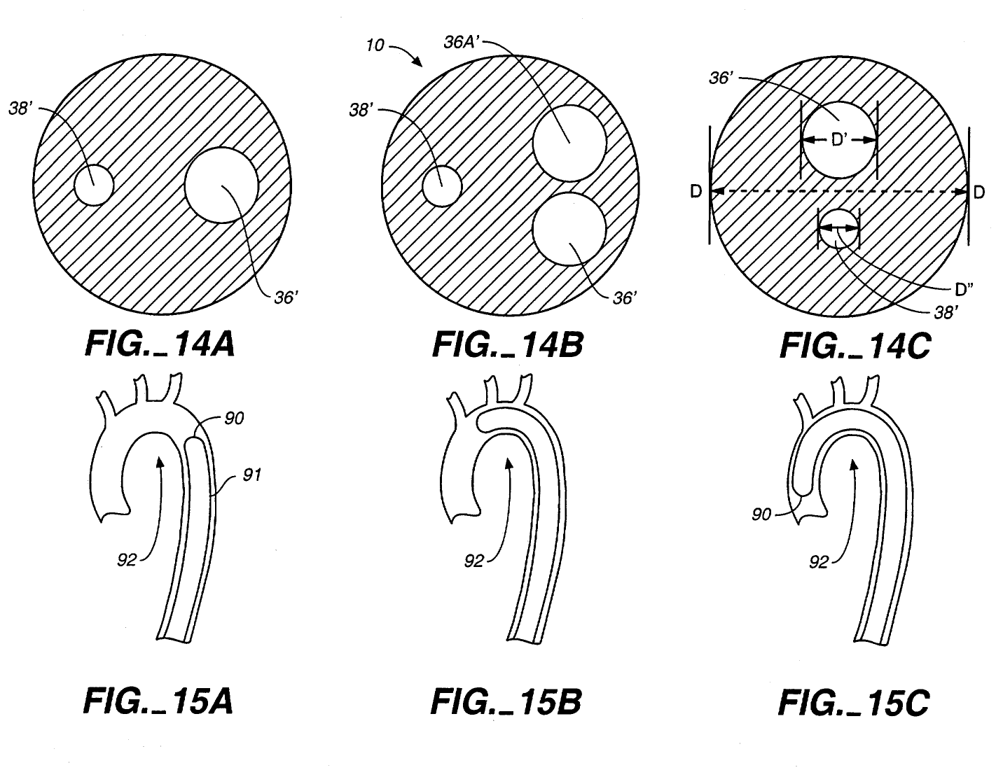

A preferred aspect of this invention is that the distal portion of the catheter is reduced in diameter from the proximal portion of the catheter. The reduced diameter of the distal portion reflects the elimination of the space needed for the blood flow in the proximal portion of the catheter. By eliminating this space, the distal portion can be viewed as having less air space and greater solid mass, a combination that reduces the likelihood that the catheter will kink and/or twist while traversing the aortic arch. Referring to FIGS. 14A and 14C, when the cross-section of the distal portion of the catheter is viewed, one can see in a preferred embodiment that there are only two lumens (also referred to as channels) 36′ and 38′ (corresponding to 36 and 38 in FIGS. 5A and 5B), one for inflating the balloon and one for transporting cardioplegia solution or a guide wire to the tip of the catheter. An additional lumen could be used for an optical cable, a pressure monitoring device or the like. In FIG. 14B, 3 lumens 36A′, 36′, and 38′ (corresponding to 36A, 36, and 38 in FIG. 5D) are shown. It is preferred that about 50% or more of the cross-sectional area would be mass (i.e., the polymer used) and less than 50% of the cross sectional area would be lumen or air. More preferred is that 60% or more is mass, and most preferred, at least 75% of the cross-sectional area is mass.

The dimensions of the distal portion and the internal lumens will be such that each will reasonably perform its functions. For example, the cross-sectional diameter of the distal portion along dotted line D-D in FIG. 14C will be about 0.15 cm to about 0.30 cm, generally less than about 0.20 cm, e.g. about 0.197 cm. The cross-sectional diameter of the larger lumen in FIG. 14C along dotted line D′-D′ is about 0.05 cm to about 0.10 cm, generally less than about 0.09 cm, e.g. about 0.085 cm. The cross-sectional diameter of the smaller lumen in FIG. 14C along dotted line D″-D″ is about 0.02 cm to about 0.05 cm, generally less than about 0.045 cm, e.g. about 0.042 cm.

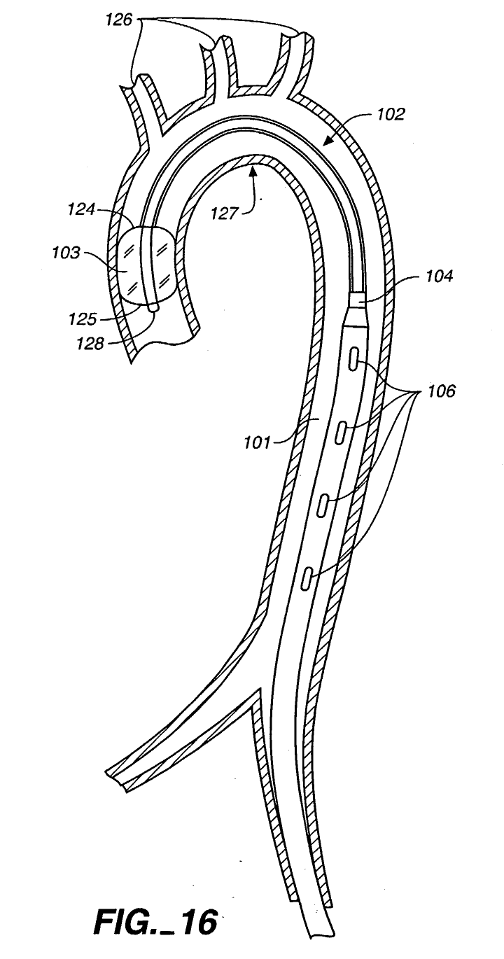

The distal portion of the catheter is flexible, is of a durometer rating that it easily bends when confronted by the aortic arch, and has a tip that is nontraumatic, i.e. the tip has a rounded or blunt (non-sharp) surface. Referring now to FIGS. 15A, 15B, and 15C (not drawn to scale and not showing the balloon) one sees the advantage of a straight, nonkinking design as discussed hereinbefore. As the nontraumatic tip 90 of the distal portion of the catheter is inserted to reach the bend of the aortic arch 92, a straight design tends to go to the upper portion of the arch. As shown in FIG. 15B, as the catheter proceeds through the aortic arch, the rounded tip 90 of the distal portion will glide guided by the top of the aortic arch and will readily flex and traverse the arch to finally be positioned with the tip 90 pointed straight down as shown in FIG. 15C. The tip of the distal portion is then ideally positioned (straight) for the balloon, which is preferably cylindrical in shape as shown in FIG. 16, to be oriented in a manner that, as the balloon inflates, forces are perpendicular to the walls of the ascending aorta. This results in an improvement in the ability of the occlusion balloon to effectively balance and equalize the opposing forces between the balloon and the wall of the ascending aorta. FIG. 16 shows an inflated balloon properly positioned using the catheter of this invention where both the distal portion and the proximal portion are shown. The distal portion is sufficiently flexible as that it will bend but not kink at body temperature. The Durometer rating will be about 60A to about 90A, preferably about 80A. In general, the length of this distal portion will be less than about 50 cm, preferably about 15-30 cm.

As shown in FIG. 16, an inflatable balloon, i.e., a non-porous sac, is integrated to the distal end of the catheter of the invention. The balloon can be inflated and distended by pumping a fluid into its interior. The balloon, when inflated, will preferably have the distal side within a centimeter or less of the tip 128 of the device. The interior of the inflatable balloon is in fluid communication with the channel 38′ (in FIG. 14A) so that the balloon can be inflated or deflated by transporting fluid through the channel to the balloon to inflate it or sucking the fluid out to deflate the balloon. The design of the balloon may be any design known in the art, such as that shown in U.S. Pat. Nos. 5,423,745; 5,516,336; 5,487,730; and 5,411,479, the pertinent parts of which are incorporated by reference. Other useful balloon components are commercially available to one of ordinary skill. It is also preferred that the distance between the proximal side 124 of the balloon and the distal side 125 (as shown in FIG. 16) be such that the surface contact with the interior wall of the ascending aorta wall be maximized. This helps ensure a tight seal to prevent leakage. This longitudinal distance between 44 and 45 may be from about 10 mm to about 50 mm, preferably about 20 mm to about 30 mm, e.g. 24-26 mm. The cross-sectional diameter of the inflated balloon will be the diameter of the patient's aorta and will vary from patient to patient. The longitudinal distance is preferably greater than the diameter, thus the balloon will preferably be somewhat cylindrical in shape. A useful filling volume of the balloon is 10 cc nominal to achieve the desires cylindrical shape (e.g. 26 mm) with a maximum inflation volume of about 35 cc. Maximum inflation pressure will generally not exceed 400 mmHg.

The design of the distal portion is such that the radial forces exerted by the tip of the catheter are less influenced by curvature or angulation of the shaft of the catheter. That is, there is a change (reduction) of structural rigidity of the catheter from the proximal end to the distal tip. This facilitates positioning of the catheter tip. The balloon integrated around the catheter can be used to position (or control position) of the catheter in the desired orientation within the aorta. This “desired” position can be a central or eccentric location. The shape, size, materials, mounting and physical characteristics of the balloon can be modified to control the desired positioning of the catheter within the blood vessel.

The forces imposed upon the wall of the ascending aorta are evenly distributed over the surface area contacted with the preferred cylindrical balloon. A spherical balloon, known in the art (e.g., the Heartport catheter model #EARC-23EAC), concentrates and directs all of the force towards a smaller area of aortic wall near the apex of its curvature. While the magnitude of the concentrated force from a spherical balloon is equivalent, a distributed force resulting from a cylindrical balloon poses fewer problems in terms of balloon stability. This is due to the fact that the cylindrical balloons tend to naturally orient the forces perpendicular to the aortic wall by distributing the force over a larger surface area.

Applicant's preferred cylindrical balloon as shown in FIG. 16 cannot “pivot” within the ascending aorta as easily as a spherical balloon because a cylindrical balloon increases surface contact with the wall of the ascending aorta and has, therefore, an increased propensity for stability. Forces that influence the balloon stability included those of the balloon against the aortic wall, those of the wall against the balloon, and those exerted by the catheter shaft (e.g., leverage and torsion). These forces will continue to search for a point of balance until it is found. Until balance is obtained the balloon will remain unstable within the ascending aorta.

Applicant's preferred balloon as shown in FIG. 16 is designed to be symmetrically integrated into the distal end of the catheter, thus providing the best opportunity to balance and equalize the opposing forces between the balloon and the wall of the ascending aorta. Asymmetrically mounted balloons known in the art (e.g., Heartport model #EARC-23 EAC), while useful, provide greater opportunity for instability due to their inability to effectively balance the opposing forces between the balloon and the wall of the ascending aorta.

Balloon taper is preferably minimized in order to maintain cylindrical profiles as shown in FIG. 16. Tapered balloons may orient the catheter tip towards the outside of the aortic arch. As tapered the balloon is then inflated the inflation axis of the balloon (perpendicular to the catheter shaft) is not oriented perpendicular to the walls of the aortic arch. This then allows the balloon to continue its expansion, which in turn may force the catheter tip into the wall of the ascending aorta possibly resulting in occlusion of the exit orifice of the cardioplegia lumen.

Transesopohageal Echocardiography (TEE) monitoring is useful to monitor balloon occlusion function, while Fluoroscopic monitoring is recommended.

While the surface of the balloon may be smooth, it may have a design on it that provides additional friction between the balloon surface and the internal surface of the aortic arch. Thus the balloon surface may have either depressions, or ridges in a design that helps maintain the balloon in position. It is preferable to have on the surface of the balloon certain ridges or bumps to provide additional friction for maintaining the position of the balloon in place and minimizing the disruption of plaque that may be present. Generally, the volume of the balloon will be about 30 to about 100 cubic centimeters, preferably about 30-40 cc. The length of the balloon from its proximal end 44 to its distal end 45 (FIG. 16) will generally be about 1.5 cm to about 7.5 cm with about 2 to 3 cm being preferred. It will need to expand sufficiently to block the ascending aorta completely so that blood does not get to the arrested heart from the cardiopulmonary machine.

The Characteristics of the Combined Distal and Proximal Portions

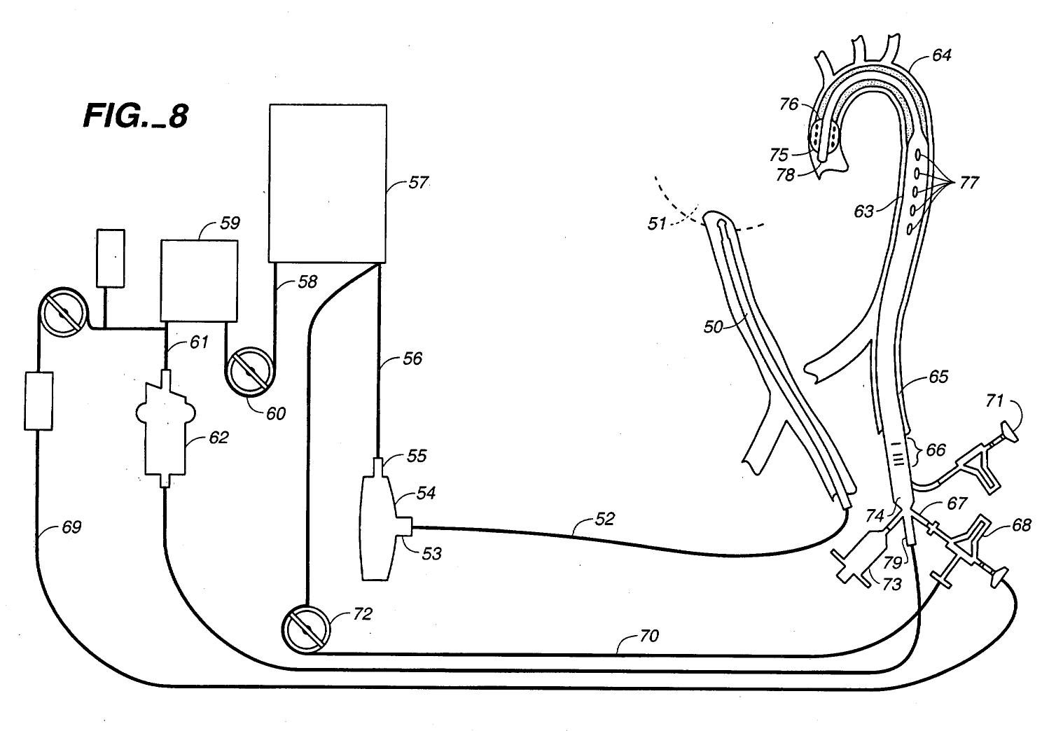

The combination of the distal and proximal portions, preferably with the obturator, comprise a device that may be a disposable, e.g., a flexible polyurethane device. An inflatable polyurethane balloon is integrated at the distal region of the distal portion of the device. The outside diameter of the proximal portion is about 14-23 French (FR: 1 FR=3 mm), preferably about 21 FR. (7 mm). It has a large central lumen for the delivery of arterial blood through multiple outlets all upstream (relative to the flow of blood through the blood-delivery lumen) of the distal portion, a lumen that runs the length of the device and exits through an outlet orifice in the tip of the distal portion in the area of the aortic root for delivery of cardioplegia solution and for left ventricular venting, and a small lumen that runs nearly the length of the device that communicates with the balloon interior for expanding and contracting the distal balloon. Radio-opaque balloon indicator and insertion depth marks may be used to aid in positioning the device. The materials used are non-pryogenic. The blood flow rates are one (1) to six (6) Liters per Minute. Maximum Recommended Blood Flow Rate is (5) Liters per Minute. See FIGS. 8, 13, and 16 for how the catheter is positioned in the aortic arch.

The catheter is made of physiologically acceptable material and is of a size suitable for insertion into a blood vessel of a mammal, particularly a human. Preferably, at least some and preferably the majority of the plurality of openings communicating with the first large channel are elongate in shape with the length of the openings being substantially parallel to the length of the catheter.

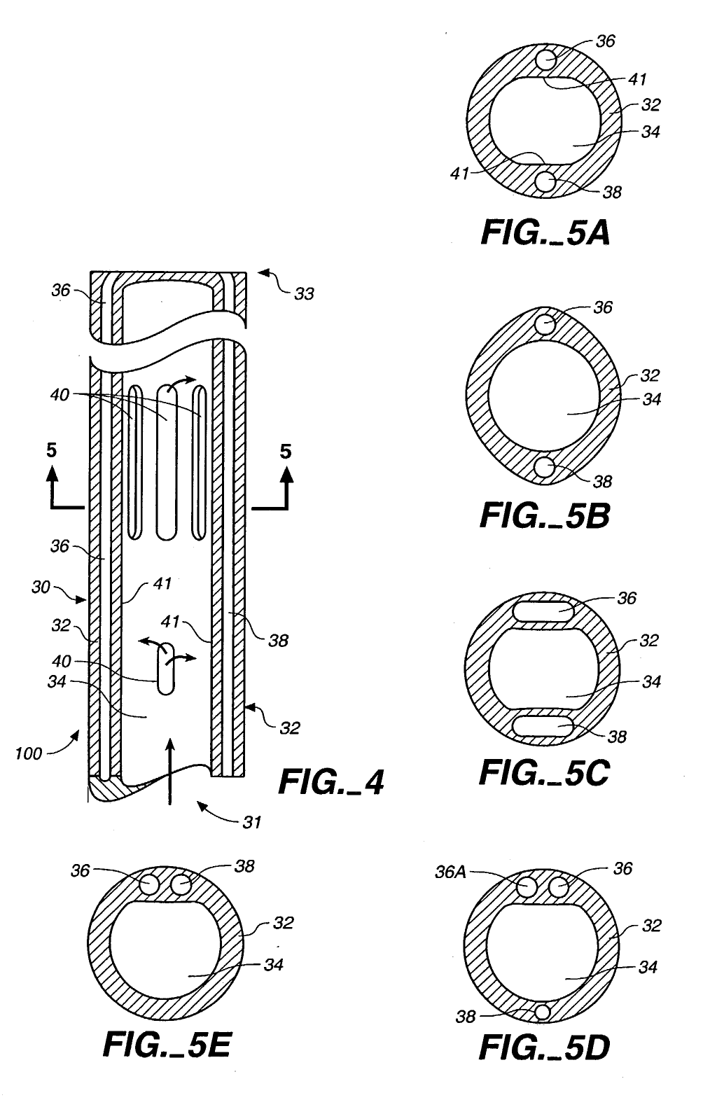

Turning now to FIG. 4, one can see a detailed representation of the proximal portion of a balloon catheter of this invention which is a cross-sectional view of a portion of the length of the catheter. The catheter (shown as a preferred 3-lumen catheter) is shown generally as 30 having a proximal end 31 and a distal end 33. The large first channel 34 is defined by the wall 32 of the catheter. The second channel 36 and the third channel 38 are shown as being integrated into the wall of the first large channel. The second and third channels are integrated with the wall 32 of the first channel 34 and are shown as having an interior wall portion 41 defining the smaller second and third channels.

Along the length of and toward the distal end 33 of the proximal portion 30 are located a plurality of openings 40 that are outlet ports for the fluid passing through the channel 34. In use, that fluid will be blood that is circulated to the arterial side of a patient in need of such extracorporeal circulation. The source of the blood will be a cardiopulmonary machine (“CPM”) with the proximal end 31 of the proximal portion 30 in fluid communication of the CPM at line 28. As will be discussed in greater detail, particularly with regard to FIG. 8, hereinafter, the catheter of this invention is preferably designed to be inserted into a femoral artery of a human patient and advanced sufficiently to allow the distal portion to be positioned in the ascending aorta. Because only the distal portion of the catheter traverses the aortic arch, the proximal portion does not have to be designed to avoid kinking. The openings 40 communicating with channel 34 are located on the proximal side (i.e., upstream) of the distal portion of the catheter that carries the balloon so that blood flows out of channel 34 through outlets 40 toward the great arteries. The openings may spread along the length of the proximal portion. See FIG. 16.

It should be noted that the total outflow capacity of the outlet ports 40 is generally greater than the inflow capacity of the blood flowing into the catheter. This will mean that total collective cross-sectional area of openings 40 will exceed the total cross-sectional area of channel 34. Thus, to calculate the collective cross-sectional area of openings 40, one determines the area of each opening and adds the area of each opening. Preferably the total area (i.e., outflow capacity) of the openings will exceed the cross-sectional area (i.e., inflow capacity) of channel 34 by at least a factor of 1.2. Having a factor of greater than about 2 is even more preferable. For example, if the radius of channel 34 is 2.5 mm, the cross-sectional area is 19.6 (2.5.times.2.5.times.3.14=19.6) and the total cross-sectional area of the openings 40 will be at least 23.6 (1.2.times.19.6=23.6), more preferably 39.2 (2.times.19.6=39.2). Preferably, each opening has a cross-sectional area of about 3-40 mm², preferably about 5 to about 20 mm². The total number of openings may be as few as 3 large openings up to about 20 openings of various shapes.

While the shape of the openings 40 may be of any appropriate shape for the outflow of blood, it is preferable that some, generally a majority of the openings are elongate in shape. While the openings may be positioned in any configuration at the distal end of the catheter, for example, the longitudinal axis of the elongate openings may be positioned substantially parallel to the length of the catheter or at a slight angle such that it forms a helical design or the length could be perpendicular to the length of the catheter. However, it is preferred that the elongate openings have the length of the opening substantially parallel to the length of the catheter. The number of openings that can be present may vary from 3 to 20 or more. By having elongate openings instead of circular openings the sheer stress on the blood is reduced by allowing the blood to flow out of the outlets more easily. The design of the openings 40 may generally be that of an oval, oblong, a rectangle, a trapezoid or some similar elongated design. In general, they will be approximately one cm to about four cm, preferably about 2.5 cm long with a width at the broadest portion of the opening no more than about 5 mm. By having a majority of (e.g., oval) openings and ensuring the outflow capacity exceeds the inflow capacity the sheer stress on the blood passing through the first channel 34 will be significantly reduced. By having the elongate openings at the distal end and maximizing the size of channel 34, the flow rate through the large channel 34 may be up to six liters (L) per minute without having adverse affect on the blood due to too much shear stress on the red cells, platelets or white cells. Having the elongate openings and proper outflow capacity also reduces the pressure drop between the proximal end where the catheter is attached to the cardiopulmonary machine and the exit at the openings 40. Generally, the pressure drop will be under 300 millimeters of mercury and preferably under 200 millimeters of mercury. The pressure drop can be further reduced by having additional holes towards the proximal end of the proximal portion but somewhere between the midpoint of the catheter and the distal end. This design is seen in FIG. 16. Preferably, the size of the outlet ports increase in size the further away from the CPM. This tends to provide a more uniform dispersion of flow.

In general, the maximum length of the multichannel catheter of this invention (including both distal and proximal portions) will be that length necessary to insert the catheter into the femoral artery of the patient and moving it up the artery to place the distal end having the balloon within the ascending aorta. Depending on the size of the patient, whether a child or an adult, the length may be from about 40 centimeters up to about 120 centimeters or more. Generally, the range will be about sixty to about one hundred centimeters with about eighty-five centimeters being an average length suitable for most people.

The outside diameter of the multichannel catheter of this invention will be such that it can be inserted and moved through the femoral artery of the patient and located in the ascending aorta as discussed above. Generally, this will have an outside diameter (OD) of no more than about 30 French, preferably of about 14 to 23 French with about 20 to 22 French outside diameter for the proximal portion fitting most patients. The French scale is a scale used for denoting the size of catheters or other tubular instruments, with each unit being roughly equivalent to 0.33 millimeters (mm) in diameter. For example, 18 French indicates a diameter of about 6 millimeters while 20 French would indicate a diameter of about 6.6 millimeters. The thickness of the wall 32 may be between about 0.2 mm to about 1.0 mm. Thus, the inside diameter of channel 34 will generally not exceed about 28.2 French, and may vary from about 14.8-22.5 French. It is found that 15 FR for the distal portion and 22 FR for the proximal portion works well.

Referring again to FIG. 4, a second channel 36 is designed to introduce a cardioplegia solution, to evacuate fluid (i.e., vent the left ventricle), or to carry a guide wire or various types of probes or for treating the heart. Thus, it has at least one opening at the distal end of catheter 30, which communicates with a corresponding channel in the distal portion of the catheter of this invention and leads to the distal tip of the distal portion of the catheter, which tip is downstream of the balloon. This allows a cardioplegia solution, a guide wire or the appropriate fiberoptic cable to be inserted into the channel and moved through the channel out exit 128 in FIG. 16 or exit 78 in FIG. 8. It also allows for a negative pressure to be applied to vent the left ventricle of the heart, if desired.

In a preferred mode of operation, the catheter of this invention is inserted percutaneously or by cutdown into the femoral artery of a patient and is threaded through the femoral artery to the ascending aorta to be positioned there. See FIG. 8. Occasionally, it may be necessary to supplement the flow of a patient's heart if it has been weakened, and this can be done by flowing oxygenated blood through the central passageway 34 of the catheter (FIG. 4) out the outlets 40 to the great arteries and other arteries in the arterial system. If an operation is to be performed on the heart, which requires arrest of the heart, the catheter is positioned so that the balloon is positioned between the coronary ostia and the great arteries as shown in FIG. 8. The balloon is inflated to block the flow of blood into the heart from outflow openings 40 in FIG. 4 or 77 in FIG. 8. Cardioplegia solution is administered through channel 36, 36′ out opening 128 (FIG. 16) or 78 in FIG. 8 to arrest the heart. Blood is then circulated through channel 34 out openings 40 (FIG. 4) or 77 in FIG. 8 to maintain circulation of oxygenated blood in the patient during the operation.

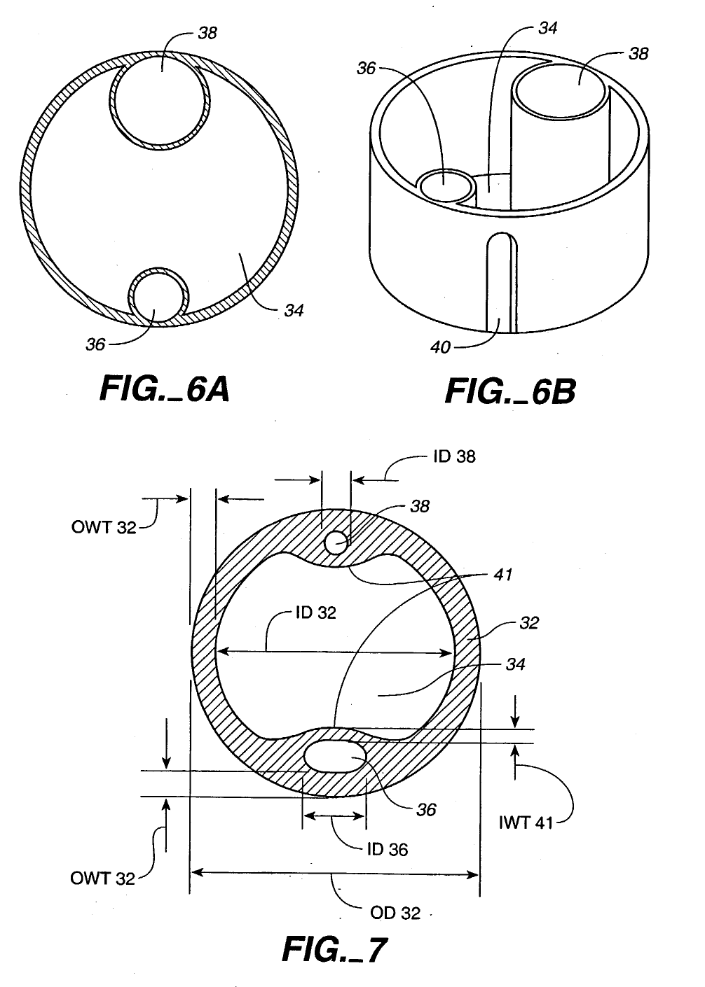

Turning now to FIGS. 5A through 5E and FIGS. 6A and 6B, one can see a cross-sectional view taken along lines 5--5 in FIG. 4. In these figures, it can be seen that the large central passageway 34 is defined by the wall 32 of the overall proximal portion of the catheter and that the channels 36 and 38 are integrated into the wall 32. They may be integrated so that they are positioned more interiorly as shown in FIG. 5A or more exteriorly as shown in FIG. 5B with cross-sectional diameters that are essentially a circle. On the other hand, in FIG. 5C, the cross-sectional of channels 36 and 38 may be elongated or oval. FIG. 5D shows a four-lumen cross-section. While the relative volumes of the small lumens are shown to be about equal, the total volume of flow available for all passageways 34, 36 (and 36A) and 38 is divided as follows. The volume for passageway 34 will make up a majority of the available volume, preferably be at least about seventy percent or more (e.g., up to about 90%) in order to achieve the advantages of this invention with the flow through passageways 36 and 38 being the minority, i.e., the remaining thirty percent or less (i.e., down to about 10%). In general, there will need to be less volume in the channel for communicating with the balloon than in the channel that is available for the cardioplegia or the fiberoptic instruments or cable. While generally, it is preferable to have the channels 36 and 38 opposed one hundred eighty degrees from each other as shown in FIGS. 5A to 5C, it may be possible to have them adjacent as shown in FIG. 5E. Having them adjacent makes the preparation a bit more difficult than having them opposed as in FIGS. 5A, 5B and 5C. FIGS. 6A and 6B show a representative cross-section and cross-section perspective view of the proximal portion of the catheter of this invention.

The ratio of the total volume of the cardioplegia channel 36 to the balloon inflating channel 38 will vary from about 1:1 to about 4:1. So, for a multichannel catheter in which about 70% of the total available volume is provided for the channel 34 and about 30% of the total available volume is provided for channels 36 and 38, channel 36 will account for about 15% to about 24% with channel 38 accounting for about 15% to about 6%. Alternatively if channels 36 and 38 collectively account for about 10% of the total available volume then channel 36 will have about 5% to about 8% while channel 38 will have about 5% to about 2%.

By referring to FIG. 7, one can see the relative proportions of the three channels of the multi-channel catheter of this invention. In the figures the abbreviations have the following meanings:

ID—inner diameter

OD—outside diameter

IWT—inner wall thickness

OWT—outer wall thickness

Summarizing the dimensions, they are as follows:

OD 32: 16-30 French (5.3-9.9 mm)

ID 32: 14.8-28.2 French (4.7-9.3 mm)

OWT 32: 0.6-1.0 French (0.2-0.3 mm)

IWT 41: 0.6-1.0 French (0.2-0.3 mm)

ID 38: 0.6-1.0 French (0.2-0.3 mm)

ID 36: 0.6-4.0 French (0.2-1.3 mm)

The catheter of this invention is able to handle a blood flow rate through the central channel 34 of about one-half up to about 6 liters per minute with the proper sizing and design. Generally, a flow of about 4.5 to 5 liters per minute is sufficient to handle the vast majority of circulatory needs required by patients having heart surgery performed. On the other hand, the flow of cardioplegia solution or drug-containing solution through channel 36 is generally about 100 to about 300 cubic centimeters (0.1-0.3 liters) per minute. The balloon inflation channel 38, which is generally smaller than channel 36, will be of a size sufficient to carry balloon-inflating fluid, e.g., saline, to the balloon. The volume of the balloon is generally about 40 cc to about 100 cc, generally about 60 cc. Thus, channel 38 is of a size sufficient to carry that volume over a short period of time, i.e., less than a minute and generally less than about 10 seconds. The volume of the balloon will be greater if the distal end of the multichannel catheter is tapered in the region covered by the balloon.

In general, the catheter of this invention will need to be flexible enough to easily be inserted up through the femoral artery to be positioned in the ascending aorta. The flexibility of the distal portion needs to be sufficient so that the catheter can bend but will not kink at body temperature. In general, this flexibility is determined by Durometer and will be in the 60A to 90A range. Generally, the Durometer reading of about 80A is preferable. It is preferable that the distal end where the balloon is located has the appropriate flexibility to allow the distal portion to transcend the aortic arch. This helps to position the catheter in the ascending aorta to ensure proper alignment of the balloon. Eliminating the “blood flow” lumen (e.g., increasing the ratio of mass:air to ≧50%) in the distal section causes increased flexibility without kinking due to increase in tolerated radius of curvature. This increased flexibility and tighter curvature radius reduces the forces exerted at the tip of the catheter which oppose curvature of the catheter. This reduces the leverage forces exerted by the catheter on the balloon (those forces which cause the balloon to twist or turn in the aorta).

Turning now to FIG. 9 one can see a more detailed description of the catheter of the invention and how it would work in conjunction with the cardiopulmonary machine. The device of the invention is generally indicated as 100 with the proximal portion being designated as 101 and the distal portion being designated as 102. At the distal portion of the device there is a balloon 103 which is integrated into the distal tip of the distal portion of the device. The distal portion is joined with the proximal portion at juncture 104 where the cross sectional diameter of the device will transition from a greater diameter of the proximal portion (for example 21 French to a smaller diameter of the distal portion (for example 15 French). An inlet 105 for the oxygenated blood from a cardiopulmonary machine is shown, which inlet can be connected to the appropriate line of the machine to receive oxygenated blood that will ultimately be channeled into the large central channel for delivery to the arteries. This channel is designated as 34 in FIGS. 4 through 7 and was discussed earlier in the application. The outlet ports 106 are shown distributed along the distal region of the proximal portion of the device. These outlet ports are to allow the blood to escape once the catheter is properly positioned as shown in FIG. 8.

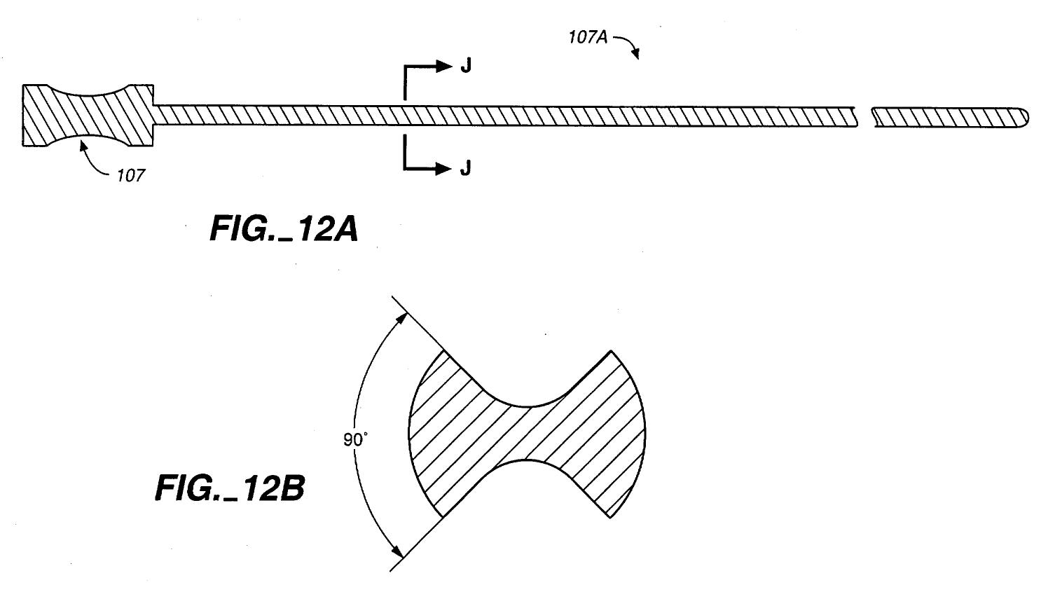

The obturator has an enlarged handle 107 attached to the stem of the obturator 107A that slidingly fits into the interior of channel 34 through entry points for the obturator 108. When the obturator is fully inserted into the channel it will extend past the furthermost outlet port 106 nearly to the juncture 104. When it is desired that blood should be delivered to the patient through port 105, the obturator is pulled out of the channel until the tip of the obturator reaches a position where blood can flow past the obturator and into the channel 34 and ultimately out the outlet port 106. The obturator is shown in greater detail in FIG. 12A where the handle 107 is shown along with the stem 107A. It will be noted that the end of the obturator is a flat, blunt, or rounded end as compared to a sharp taper. This is to avoid damaging the interior of the channel particularly the end of the channel. The cross section of the obturator shown in 12A at lines JJ is shown in 12B. The cross section will correspond approximately to the cross section shown in FIGS. 5A-5E, 6A-6B or 7. The obturator fits slidingly and snugly within the large blood carrying channel and performs several functions. The tip of the catheter device of the invention is inserted into the femoral artery while blood is being pumped by the heart. As the device is inserted and reaches a point where the furthest outlet port 106 is inserted into the artery, if the obturator is not in place blood will flow into that port and out of the other ports and into the operating arena if all of the ports are not inserted at the same time. By having the obturator blocking the ports, the flow of the blood through the large channel from the heart is prevented. Once the device is inserted so that the most proximal outlet port 106 is fully located within the artery the obturator can start being withdrawn until the tip of the device is properly positioned to be between the coronary ostia and the great arteries such as the brachiocephalic artery. Once the balloon is positioned appropriately it can be inflated by sending a fluid such as saline through line 117 and entry port 118. A valve 119 is situated at the entry point port to allow the saline to be turned on or off. The valving is such that fluid may be inserted through port 118 and withdrawn from that port or input through line 120.

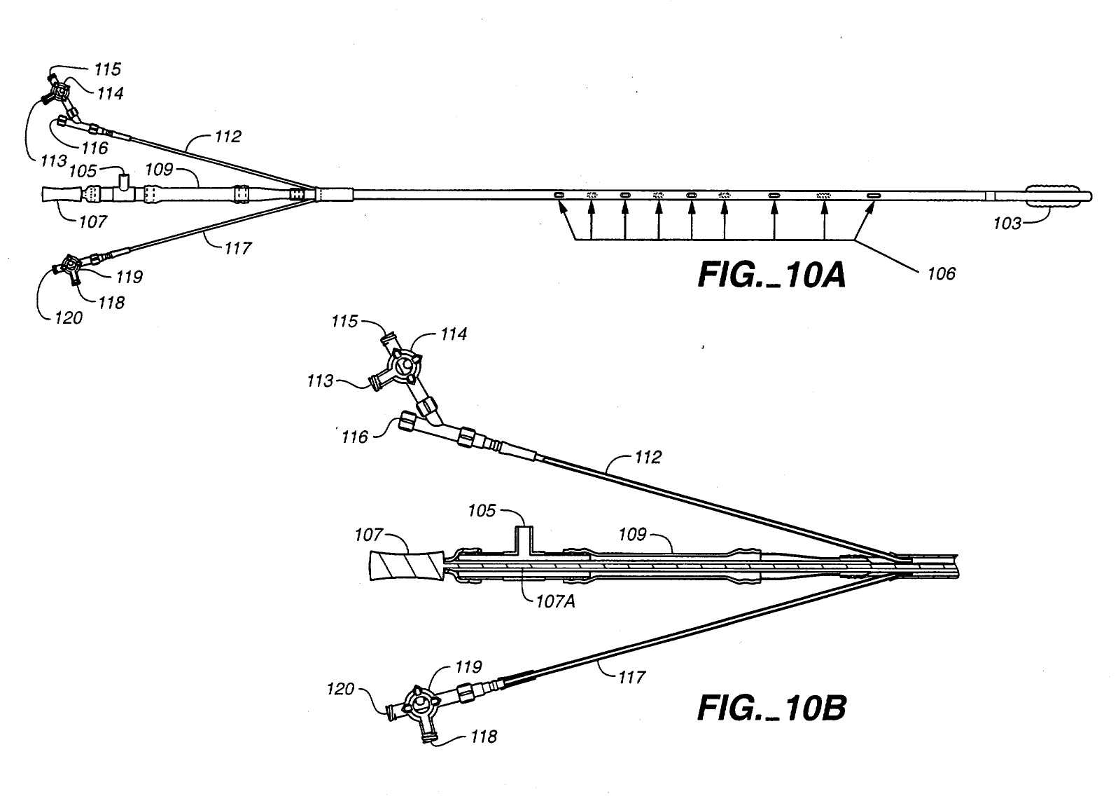

Once the balloon is in place the cardiaplegia solution is pumped through line 112 through the interior of the device and out through the tip as shown in FIG. 8. The cardiaplegia coming out of tip 78 will reduce the rate of beating of the heart or stop the beating completely. The cardiaplegia can be sent through inlet 113 through valve 114. If desired valve 114 can be adjusted so that the heart could be vented through outlet 115 by providing a slight vacuum to pull excess blood out of the area. If desired a guidewire may be inserted through line 116 through line 112 and through the internal channels 36 and 36′. Once the obturator has been fully withdrawn, CPM blood will flow into the blood carrying channel through port 105 and through flexible connection line 109 which may be connected by a slip fit or twist fit 110 and 111. The cardiaplegia has slowed the beating of the heart and the balloon has been properly inflated to prevent any CPM blood from getting to the heart. As CPM blood flows to the great arteries and the rest of the body, the surgeon can perform the appropriate surgery on the heart. To aid the surgical team in the proper insertion of the device and to aid in positioning the device, distance markings designating the distance from the tip 128 of the device are used these markings are shown in FIGS. 9 and 11 as numbers 121. Thus in FIG. 11, VII, for example, will indicate that it is 70 cm from the tip of the device, VI would mean 60 cm from the tip, V would mean 50 cm from the tip. In addition there may be a warning indicator 123 that may indicate that the indicator is about 45 cm from the tip and a few centimeters from the nearest outlet port 106. In addition at the junction of lines 109, 111 and 112, the junction being shown as 122 there is a place for a serial number to indicate the number of the device that has been manufactured. Further details of FIG. 9 can be seen in FIGS. 10A and 10B where like numerals refer to like parts of the invention.

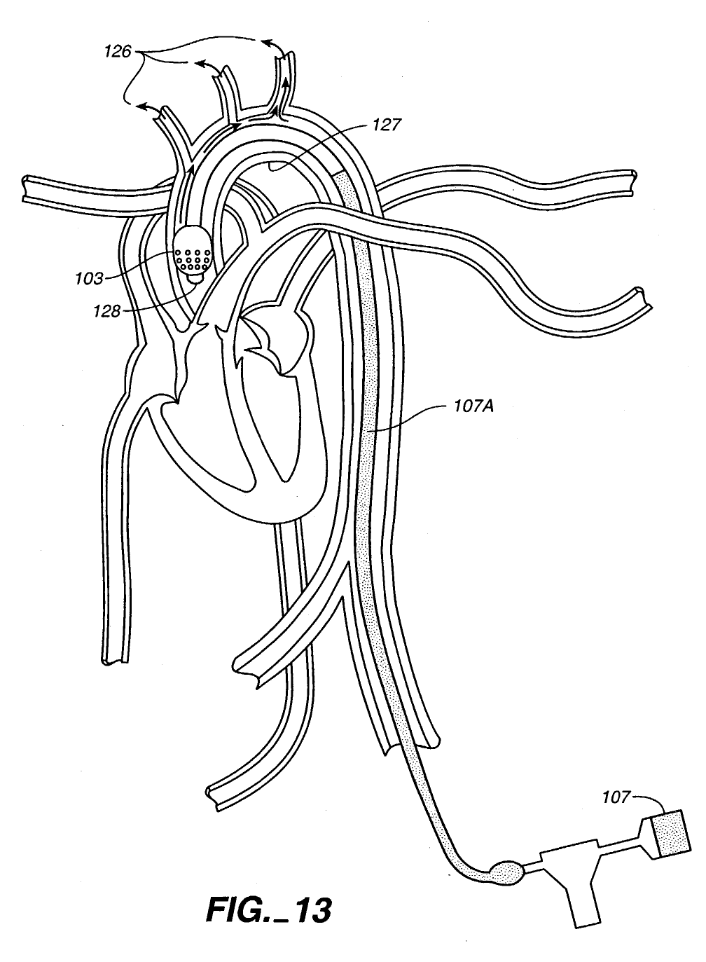

Turning now to FIG. 16 one can see the device placed in the aortic arch with the balloon 103 blocking the ascending aorta and situated between the coronary ostia and the great arteries 126. The distal portion 102 of the device is shown arched over the aortic arch 127. The proximal portion of the device 101 is shown to be positioned relatively straight with the juncture 104 between the proximal and distal portions as shown. FIG. 13 is a simplified version showing a device in which the distal portion of the device is not reduced in diameter as compared to FIG. 16. In the figure the obturator is shown as having handle 107 and stem 107A which is shown as the shaded portion in the figure. One can see that the obturator does not extend the full length of the device but instead the distal portion of the device that fits over the aortic arch does not have the obturator in and does not have the large first channel. Again the reason for this is to minimize the likelihood of kinking in the portion that goes over the aortic arch.

In performing open heart or least invasive cardiac surgery, generally, it is necessary to do an angiogram by placing an angiogram catheter up the femoral artery and positioning it in the ascending aorta. Based on the length of the angiogram catheter balloon placement position can be determined, the multi-channel catheter of this invention has markings indicating its length measured from the distal end to various distances near the proximal end so that the physician knows exactly how far to insert the catheter of this invention. Having that information indicated on the catheter makes it easier for the physician to do the insertion and also reduces the need to use fluoroscopy to properly insert the catheter. On the other hand, if a angiogram catheter measurement is not done before inserting the catheter of this invention, an ultrasound probe may be used to position the catheter of this invention where the catheter of this invention carries a detectable beam on the tip of the catheter. Alternative methods may be employed for positioning the catheter, such as guidance by fluoroscopy or echocardiography, fiberoptic visualization through the catheter, magnetic or electronic guidance, or other means of insuring proper placement.

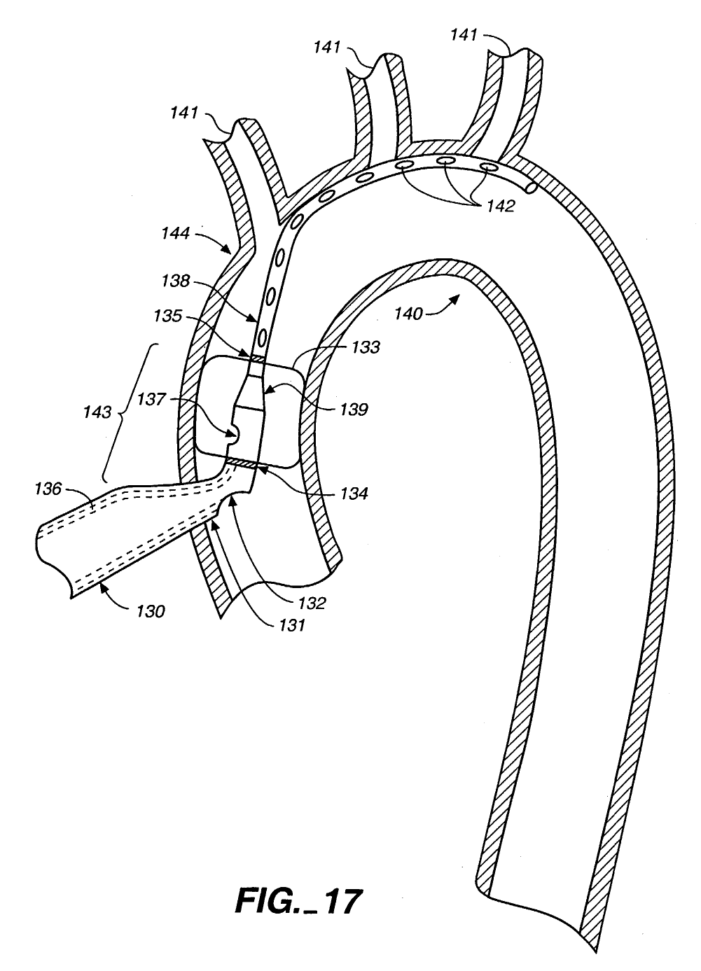

An alternative design for a multi-lumen catheter of this invention is shown in FIG. 17. Here the parts of the catheter are slightly reversed from what the previous discussion has set forth. Here a balloon catheter for delivering blood to an animal, particularly a human, is positioned to block the aortic arch between the great arteries 141 and the coronary ostia, not shown, by entering via the base of the aorta 144. A proximal blood transport section of a multi-lumen catheter of this invention is shown as 130. It has distal and proximal ends and is conjoined with the proximal end of the distal blood delivery section 138 of the device. In the proximal blood transport section 130, a first blood transport lumen is defined by the surrounding wall extending the length of the blood transport section and is open for communication at its distal end as well as at its proximal end. At the proximal end a cardiopulmonary machine, not shown, is attached for circulating extracorporeal blood. A second lumen extends the length of the blood transport section and is parallel in the first lumen but independent of it. This lumen is generally used for transporting cardioplegic solution and is open at its distal end as shown as 132 to allow cardioplegia solution to exit 132 for delivery to the base of the aorta and to the heart to slow or stop the heart. A third lumen is located in the proximal blood transport section of the device. The third lumen is independent of and parallel to the first and second lumens and extends the length of the three-lumen portion. It is open at the distal end and communicates with the interior of the inflatable balloon 133, which is integrated into the distal region of the blood transport section. One can see that the balloon 133 interior communicates with outlet 137 so that it can be inflated or deflated. The balloon is integrated at the end of the distal section of the blood transport section having bonds 134 and 135 proximal and distal to the CPM. The lumen leading to the interior of the balloon is shown as 136 by a dotted line indicating that the lumen is interior to the proximal blood transport section. The distal blood delivery section 138 is shown as having ports 142 distributed along the length of this section. This section is in communication with the first blood transport lumen of 130. It can be seen that at the distal end of the blood transport section 130 there is a transition zone indicated as 143 where the transition is from three to two-lumens, generally shown at 131 and from two to one lumen. The distal blood delivery section 138 with the blood outlet ports 142 extends distal to the balloon 133 and is positioned within the aortic arch 140. Generally, this will extend under the great arteries 141. As discussed herein, the cross-sectional area of outlet ports 142 will be greater than the cross sectional area of the blood transport lumen coming from the transport section 130 and continuing on to 138. As discussed hereinbefore, by having outlet ports along the length of the blood delivery section, the shear forces on the blood are minimized. By having this particular design as in the other design aspect of this invention, one can ensure the antegrade flow of blood in the patient's system. Generally, the device will be inserted through a trocar in the chest area to ultimately be inserted at the base of the aorta 144. Preferably, in the proximal blood transport section 130 the second and third lumens for cardioplegia and delivery of fluid to the interior balloon are positioned to about 180° opposite each other. As discussed herein before, it is preferable that the balloon 133 when inflated takes a cylindrical shape and has the size characteristics discussed herein. In determining the cross sectional diameter of the sections of the device, the proximal portion 130 will have a preferred cross sectional diameter of about 20-22 French while the distal portion will be about 14-16 French. It will be noted that the distal blood delivery section 138 is at a slight angle to the proximal blood transport section 130. This angle can be anywhere between 90-125° but preferably is at an angle of about 110-120°. Thus, the angle at the transition zone 143 will be for example 115°, the angle being formed by the longitudinal axis of the blood transport section 130 relative to the longitudinal access of the proximal portion of the distal blood delivery section 138.

The material which is used to manufacture the multichannel catheter of this invention may be any material that is physiologically acceptable, that is, it is made of a material that will not have an adverse effect on the patient when used in the manner in which it is intended. Generally this will require the use of biocompatible material (i.e., the body will not react with it) for preparing the catheter of this invention. In addition, the material that is used must possess sufficient stability and flexibility to permit its use in accordance with the process of the invention. Various biocompatible polymers may be used. A polymer that is particularly valuable for preparing the catheter of this invention is polyvinyl chloride (PVC) blood tubing, that has been plasticized. Preferably the plasticizer which is used in the PVC is trioctyl trimellitate (TOTM) while the standard plasticizer di-(2-ethyl hexyl) phthalate (DEHP). TOTM plasticizer is less extractable than DEHP and produces a better blood response. Suitable PVC resin is available from Dow Chemical Corp., Midland, Mich., or Polymer Technology Group (P.T.G.) Inc., Emeryville, Calif. Another polymer that is useful for preparing the multichannel catheter of this invention is medical grade polyurethane. Other polymers may be prepared based on a family of polysiloxane-containing copolymers termed surface modified additions (SMAs). These copolymers may be blended with the base polymer before processing or coated on the blood contacting surface. When blended with the base polymer the SMA will migrate to the polymer surface resulting in a high concentration of the SMA of that surface, which has fewer adverse reactions with the blood that contacts it. When coated, device surfaces are pure SMA. High surface concentration of the SMA are responsible for the improved biocompatibility of extracorporeal circuit components. Plasticized PVC is particularly useful as the base polymer. A further description of these polymers is given in an article entitled “Surface Modifying Additives for Improved Device-Blood Compatibility” from ASAR Journal 1994 M619-M624 by Chi-Chun Tsai, et al. The article is incorporated herein by reference. Such polymers are available from P.T.G. Corp.

Other useful polymers include polyurethane-urea biomaterials that are segmented polyurethane (SPU) some of which have surface-modifying end groups (SMES) covalently bonded to the base polymer. These are described by Ward, et al. in an article entitled “Development of a New Family of Polyurethaneurea Biomaterials” in Proceedings From the Eighth Cimtec—Forum on New Materials Topical Symposium VIII, Materials in Clinical Applications, Florence, Italy, July, 1994. See also U.S. patent application Ser. No. 08/221,666, which is incorporated herein by reference.

Sometime the blood interacts with artificial surfaces of polymers in such a way that the blood coagulates on the surface creating thrombi. These thrombi can block the catheter or blood vessels, preventing the blood from flowing and causing oxygen depletion and nutrient starvation of the tissues. Thus the surface of the polymeric material used for the multichannel catheter of this invention should not give rise to thrombus formation. An anti-thrombotic agent can be used to prevent the clots from forming. Some of the blood polymer interactions are discussed in article entitled “Biomaterials in Cardiopulmonary Bypass” found in Perfusion 1994; 9: 3-10 by James M. Courtney, et al.

Polymer modifications that permit an improvement in blood compatibility while maintaining acceptable levels of other fundamental properties include the treatment of surfaces with protein, the attachment of anti-thrombotic agents and the preparation of biomembrane-mimetic surfaces. The preferred anti-thrombotic agent is the anti-coagulant heparin, which can be attached ionically or covalently. Preferably it is attached covalently.

An additional factor to consider in preparing the catheter of this invention is the relative roughness of the blood-contacting surface. Excess surface roughness has deleterious effects on blood flow through the catheter and should be avoided.

Another article that discusses the factors relating to compatibility of surfaces contacting blood is entitled “State-of-the-Art Approaches for Blood Compatibility” from Proceedings of the American Academy of Cardiovascular Perfusion Vol. 13, January 1992, pages 130-132 by Marc E. Voorhees, et al.

Uses of the Catheter of This Invention

The catheter of this invention may be used in several different ways. For a condition in a patient that needs supplementary extracorporeal blood circulation because of insufficient circulation from his or her own heart, the catheter may be introduced via a femoral artery, positioned as appropriate and attached to a cardiopulmonary bypass machine to circulate blood through the large central channel 34 and out openings 40. When appropriately positioned with the distal end of the catheter in the ascending aorta, a fine fiber optic cable may be threaded through second channel 36 to examine the aortic area of the heart. If it is determined that a heart operation is necessary, the balloon may be inflated through channel 38 to block the ascending aorta, cardioplegia solution may be administered through channel 36 to arrest the heart, and oxygenated blood from a cardiopulmonary machine is pumped through channel 34 and openings 40 into the arterial pathway of the patient's circulatory system. Thus, the device of this invention may be used in cardiovascular surgery in general or various heart examinations or treatments of artery and valvular disease. Cardiovascular surgery is meant to include surgery to the heart or to the vascular system of a patient. The catheter is particularly useful in cardiac surgery, whether open chest surgery or minimally invasive heart surgery, particularly CPB. Such surgery may include, but are not limited to, the following:

1. Coronary artery revascularization such as:

(a) transluminated balloon angioplasty, intracoronary stenting or treatment with atherectomy by mechanical means or laser into the coronary arteries via one lumen of the catheter, or

(b) surgical mobilization of one or both of the mammary arteries with revascularization achieved by distal anastomoses of the internal mammary arteries to coronary arteries via a small thoracotomy.

2. Any atrial or ventricular septal defect repair such as by:

(a) “closed” cardioscopic closure, or

(b) closure as in “open” procedure via a thoracotomy or other limited access incision.

3. Sinus venosus defect repair similar to above.

4. Infundibular stenosis relief by cardioscopic techniques.

5. Pulmonary valvular stenosis relief by cardioscopic techniques.

6. Mitral valve surgery via thoracotomy.

7. Aortic stenosis relief by the introduction of instrumentation via a lumen in the aortic catheter into the aortic root.

8. Left ventricular aneurysm repair via a small left anterior thoracotomy.

A significant advantage of the unique multichannel catheter of this invention is its ability to be adapted to be used in accordance with the needs of a patient. For example, a patient with symptomatic coronary artery disease undergoes a diagnostic evaluation to determine the type of treatment that best suits that patient's condition. As a result of the evaluation, the physician may recommend surgical treatment, interventional cardiology treatment or some alternative treatment. Interventional treatment may include percutaneous transluminal coronary angioplasty, atherectomy or the use of a stent to keep the vessels open. Alternative treatment may include the use of a laser or myoplasty.

If additional treatment is recommended, the multichannel catheter of this invention is particularly valuable in the further evaluation to determine the condition of the patient, the type of treatment recommended and the type of drugs that might be useful to administer to the patient. Thus, in using the multichannel catheter of this invention, the catheter is inserted into a femoral artery by percutaneous puncture or direct cut-down. The distal end of the catheter, which carries the balloon, is inserted first and moved through the femoral artery to be positioned in the ascending aorta as discussed in more detail herein. Generally, the catheter will have an obturator associated with it, which is used as discussed under the “Representative Use of the Catheter.” Initially, the physician performing the work may wish to introduce instruments through the channel (36 in FIG. 4) or other probes to allow observation or measurement of the internal condition of the artery, aortic arch and/or aortic semilunar valve. A cardioscope, an electrophysiology probe, a transmyocardial revascularization probe, a radiation probe, or the like may also be inserted through channel 36. Once observations are made concerning the condition of the heart and associated arteries, the physician can then take additional steps. For example, it may be desirable to administer a biologically active fluid directly to the heart or aorta using an appropriate liquid composition containing an active entity appropriate for the patient's condition. The active entities in such a biologically active fluid include drugs (particularly those having cardiovascular effect) that are pharmaceutically acceptable small organic molecules, small polypeptide molecules, larger polypeptide molecules, and even a DNA or RNA that may be useful for gene therapy. Examples of useful molecules include those useful as antianginals (e.g., organic nitrates, calcium channel blockers, .beta.-adrenergic antagonists) antihypertensive, antiarrhythmics, antihyperlipoproteinemias, myocardial contractile enhancers, antiatherosclerotic agents, and the like. Such fluids especially for cardioplegia can best be delivered through channel 36 in FIG. 4, but alternatively can be delivered in the fluid used to inflate balloon 42 through channel 38 in FIG. 4. In the latter case, the material used for the balloon would be semipermeable to allow the drug to diffuse through the balloon membrane. A drug having lipid-dissolving characteristics can be delivered through the balloon membrane. Alternatively, it may be useful to deliver such an active agent by adding it to the cardiopulmonary machine reservoir.

Once the catheter is in place as shown in FIG. 16, and observations regarding the internal conditions have been made, the physician then can move on to the next steps. For example, least invasive surgery, as discussed in U.S. Pat. No. 5,452,733, may be performed on a beating heart with no initial cardiopulmonary support, i.e., no blood would flow through the would continue to function. If at any time, the physician would decide that cardiopulmonary support would be needed, supplemental blood flow from a cardiopulmonary (heart/lung) machine could be started and work could be continued with a beating heart or a fibrillating heart. Once a decision is made to completely arrest the heart, cardioplegia solution is delivered to the heart through the channel 36 after balloon 42 is inflated to block the flow of blood to the heart from the cardiopulmonary machine. As described, the multichannel catheter of the invention can be used in least invasive surgical procedures as well as open chest surgery.

The multichannel catheter of this invention is particularly useful in performing heart surgery where the heart is arrested using a cardioplegic solution and blood is circulated to the patient via a cardiopulmonary bypass machine. In this case oxygenated blood is circulated through the large channel of the catheter of this invention. The introduction of negative pressure on the venous drainage system may be used to enhance venous drainage and reduce the need to vent the right side of the heart. Generally, the negative pressure may be maintained at the vena cavae regions (superior and inferior) using a centrifugal pump attached to a standard femoral venous cannula. A system for performing such a process is depicted in FIG. 8.

In general, the process for performing surgery on a mammal's heart comprises a sequence of steps. A single femoral access cannula is inserted into the mammal's femoral vein to position it so the distal open end of the cannula is adjacent the vena cava region of the mammal's heart and the proximal end of the cannula is attached to a cardiopulmonary bypass machine through a centrifugal pump wherein the cardiopulmonary bypass machine comprises a blood oxygenation means fluidly connected to the centrifugal pump. At about the same time a multichannel catheter of this invention is inserted into a femoral artery preferably having an obturator associated therewith, as discussed hereinafter.

The multichannel catheter is positioned within the subject's blood circulatory system such that the distal end of said catheter is positioned in the ascending aorta such that the first channel openings are located near the great arteries, the inflatable means is located on the cephalid side of the aortic valve and the distal end of the second channel is located proximate the aortic valve and downstream of the inflatable balloon.

Next, a source of oxygenated blood from the cardiopulmonary machine is connected to the proximal end of said first (blood-carrying) channel of the catheter and a source of cardioplegia fluid is connected to the proximal end of said second channel. A source of fluid is connected for inflating said inflatable means to the proximal end of said third channel and the inflatable means is inflated to block the flow of blood to the heart.

Cardioplegia solution is pumped into the heart to arrest the mammal's heart and oxygen-rich blood is pumped through said first channel out the first channel openings upstream of the balloon at a rate sufficient to maintain the subject's metabolism and perfusion while at the same time oxygen-depleted blood is removed from the mammal's vena cavae regions through the femoral vein cannula by applying a negative pressure using the centrifugal pump. The physician can then perform a surgical operation on the heart as needed and said subject is maintained as needed.

Referring to FIG. 8, the femoral vein is accessed percutaneously or by cut down using the appropriate size standard femoral access cannula 50 (such as an Research Medical Inc. #TF-030-050). This cannula conducts de-oxygenated venous blood from the vena cava 51 to PVC tubing 52 (e.g., 0.5 inch inner diameter). This tubing is attached to the negative pressure (inlet) port 53 of a centrifugal pumping device 54 (such as the St. Jude Medical #2100CP); the positive pressure (outlet) port 55 of the centrifugal pumping device is connected via tubing 56 (0.5 inch ID PVC) to a venous reservoir system 57 (such as the COBE Cardiovascular, Inc. VRB 1800). This configuration pulls blood from the vena cava 51 to the venous reservoir 57. Utilization of negative pressure in this manner to provide venous blood return eliminates the need to “vent” or empty the right heart. By using a centrifugal pump that reaches about −20 to about −50 mm of mercury (mm Hg), a sufficient negative pressure is maintained. The use of a closed reservoir system is preferred to eliminate air/blood interface and associated blood trauma. The venous blood exits the reservoir through tube 58 (e.g., ⅜ inch ID PVC tubing) using pump 60. This tube 58 is connected to an oxygenator/heat exchanger means 59 (such as the COBE Cardiovascular, Inc. model #CML DUO #050-257-000) to oxygenate the oxygen-depleted blood. The blood will be pumped through the membrane/heat exchanger by a roller pump device 60 (such as the COBE Cardiovascular, Inc. model #043-600-000). The oxygenator will oxygenate the blood and the heat exchanger will regulate blood temperature. The oxygenated arterial blood will exit means 59 through tube 61 (such as ⅜ inch ID tubing), pass through an arterial filter 62 (such as a COBE Cardiovascular, Inc. Sentry #020-954-000) and be delivered into the femoral artery via the invention multichannel catheter 63. Preferably, all blood contact components are surface modified to reduce blood trauma, patient inflammatory response and requirements for patient anticoagulation.

The invention femoral artery catheter 63 provides flow of oxygenated blood to the aorta 64. The invention catheter 63 is introduced into the femoral artery 65 percutaneously or by cut down. The invention catheter 63 can be introduced alone or utilizing a guide wire and stylet. The stylet provides assistance in allowing the device to transcend the aortic arch. Accurate positioning of the balloon will differ from other positioning methods by utilizing measurement of the cardiac catheterization catheter. The appropriate distance will be determined and indicated on the femoral artery catheter 63 prior to insertion; the distance indicator markings 66 will provide simple and accurate balloon positioning. Accurate positioning of the balloon tip may also be enhanced or verified using visualization by transesophogial echo or fluoroscopy.

The invention catheter provides a flow of oxygenated blood to the aorta as part of the cardiopulmonary bypass process. The catheter is of a length sufficient to extend from the insertion point in the femoral artery to the ascending aorta as shown in FIG. 8, which length will vary depending on the size of the patient, as discussed hereinbefore. The catheter has a proximal end 74 and a distal end 75. The catheter has an inflatable balloon 76 located on the proximal side of the distal tip 78 for fixing the catheter within the ascending aorta. A channel extends the length of the catheter to the balloon with an outlet port that communicates with the balloon interior so that the balloon can be filled with a fluid from a syringe-type inflation device 73 to occlude the ascending aorta as discussed herein. The catheter also has (a) a blood delivery channel extending from the proximal end 74 to outlet ports 77 upstream of the balloon for delivering oxygenated blood and (b) a channel extending through the entire cannula with an outlet port at distal tip 78 for a guide wire and/or delivering a cardioplegia solution to the heart through stopcock 68 into inlet port 67 and from line 69. Changing the position of the valve in stopcock 68 to connect with line 70 and providing a negative pressure by roller pump 72, allows for the venting of the left ventricle by pulling fluid from the left ventricle through the semilunar valve through opening at tip 78.

In using the catheter shown in FIG. 17 the balloon catheter as described in the discussion of FIG. 17 is inserted into the patient through the patient's aortic artery towards the root to position the balloon catheter so that the balloon is in the ascending aorta between the patient's coronary ostia and the great arteries 141. The blood delivery extension 138 is positioned to traverse a portion of the aortic arch as shown in FIG. 17. The balloon 133 is expanded to substantially block fluid communication between the patient's heart and the aorta. Cardioplegia is provided through the lumen to exist 132 so that the cardioplegia is delivered to the heart to slow or stop the heart. The cardiopulmonary machine is then circulated through the blood transport section 130 and to the blood delivery section 138 outlet ports 142 to the patient's aorta 144 and connected arteries. Finally, the cardiovascular surgery is performed on the patient as required and the process is then reversed with the balloon being deflated, cardioplegia stopped, and the device is withdrawn. The patient's heart is revived in accordance with the usual procedures.

How to Make the Catheter

In general, the catheter is produced by introducing, e.g., 3 or 4 single lumen extruded tubings into a molded manifold which merges each of the single lumens (3) into the mulitlumen extrusion. See FIGS. 5A-5D and 14A-14C. The multilumen extrusion of the proximal portion is fused or bonded to the distal multilumen extrusion using mandrels which prevent closure of the continuing lumens. Thus, a continuing lumen running the length of the device consists of, e.g., channel 38 of FIGS. 5A, 5B or 5E, communicating with channel 38′ of FIGS. 14A or 14C. Another continuous lumen would consist of channels 36 of FIGS. 5A, 5B, or 5E communicating with channel 36′ of FIGS. 14A and 14C. Alternatively, in the case of FIGS. 5D and 14B, channels 38, 36 and 36A communicate with 38′, 36′,and 36A′. The balloon is fused or bonded onto the distal portion of the multilumen tubing which is designed to transcend the aortic arch.

The proximal portion of the multichannel catheter of this invention is prepared using any technique that provides the multichannel catheter herein described. Preferably the second and third channels are integrated into the wall of the first channel. This may be done by forming the channels separately then conjoining them, i.e., by gluing or other means. However, the multichannel catheter may be made through a mandrel-dipping technique, or preferably a continuous extrusion process. Extrusion involves forcing a fluid polymer material (as discussed above) through a suitably-shaped die to produce the cross-sectional shape, such as that depicted in FIGS. 5A, 5B, 5C, 5D, 5E, and 6 or other suitable shape as described herein. The extruding force may be exerted by any standard means known in the art such as by a piston or ram or by a rotating screw, which operates within a cylinder in which the polymeric material such as PVC or polyurethane is heated and fluidized. The fluid material is then extruded through the die in a continuous flow. The extrusion head will have a multitubular die to provide a continuous multichannel catheter, essentially as described herein. Using a mandrel-dipping technique, a mandrel having the desired size and cross section design is dipped in or drawn through a fluid polymeric material so that the mandrel is coated with the polymer. The polymer is then dried on the mandrel and removed to give the desired design. This technique may be done at commercial manufacturers, e.g., Extrusioneering, Temecula, Calif. and others.

Once the proximal portion of the multichannel catheter is formed, whether by extrusion or mandrel-dipping, it is cut to suitable lengths and treated to provide the further characteristics of the product to make it operable. Such treatment may occur in any particular order. For example, a plurality of openings (40 in FIG. 4 or 68; 77 in FIG. 8; 106 in FIGS. 9, 10A, 11, and 16) are formed near the distal end of the proximal portion of the catheter communicating with said first channel. These openings are made in conformance with the designs discussed herein, and thus are preferably elongate in that the longitudinal axis of the elongate design may be helical or orthogonal, but is preferably substantially parallel to the longitudinal axis of the catheter itself. The openings may be provided by suitably cutting or punching the elongate design into the wall of the catheter. The design is approximately oval, rectangular, or the like with the length of the opening being about a size discussed hereinbefore. The width of the opening will be such it will not weaken the structural integrity of the distal end of the proximal portion of the catheter. FIGS. 8, 9 and 10 present various configurations for the positioning of the openings. Optionally, additional openings communicating with the first channel may be provided along the length of the catheter positioned between approximately the middle of the catheter and the elongate openings near the distal end. The openings are useful in reducing the pressure drop between the proximal end of the catheter and the distal openings to help reduce the sheer stress on the blood.

The distal portion of the catheter is similarly extruded to give a length having a cross-section show in FIGS. 14A, 14B and 14C. The openings of the distal portion (e.g., 36′ and 38′ of 14A) that correspond to openings of the proximal portion (e.g., 36 and 38 of FIG. 5A) are aligned, mandrels are positioned to prevent a closure of the communicating lumens, and the distal and proximal portions are fused or bonded or otherwise permanently conjoined.

An inflatable means, i.e., a balloon, is integrated into the distal end of the catheter such that the interior of the balloon communicates with the outlet of the balloon communicating channel to allow fluid to flow through the lumen and to the interior of the balloon. In general, this may be integrated by positioning a balloon having an opening corresponding to the opening to the appropriate channel and adhering the balloon to the distal end of the catheter. This adherence may be performed by using a suitable glue, solvent bond, light sensitive weld, or other suitable means known in the art for this purpose. The material used for the inflatable means may be any suitable biocompatible material that is capable of being inflated and deflated a plurality of times. Polyurethane-based biocompatible polymers are preferred. These are described in the aforementioned article by Ward, et al.

Preparing the device shown in FIG. 17 is similar to the method as discussed hereinbefore. One of ordinary skill in the art by reviewing the methods previously described can apply those to the device shown in FIG. 17.

### EXAMPLE 1

This example provides a step-wise description of a representative use of the device of this invention that is inserted via the femoral artery.

1. Before a device of this invention is used in a patient in need of surgery suggested herein, the patient is screened to determine if surgery and usage of the device is appropriate. Preoperative screening of patients includes evaluation by sufficient methods (such as clinical examination, segmental doppler examination, aortogram) to exclude those with aortoiliac disease or anatomy that would preclude safe introduction of the balloon catheter into the aorta from a femoral artery.

2. The patient is anesthetized, positioned, prepped and draped for cardiovascular surgery requiring cardiopulmonary bypass. Arterial pressure is monitored using a right and left brachial or radial artery pressure monitoring line, which should be continuously simultaneously monitored, sudden differences in right and left pressure may indicate balloon blockage of the innominate artery. Intraoperative monitoring with transesophageal echocardiography (TEE) is required. Fluoroscopy with capability of imaging the thoracic aorta may be used but is not an alternative to intraoperative monitoring with (TEE). The aortic arch and ascending aorta should be evaluated for the presence of atherosclerotic disease associated with luminal projections, a contraindication for use of the catheter of this invention. The aortic valve should be inspected for significant insufficiency, a contraindication for delivery of cardioplegia in the aortic root with the balloon catheter of this invention.

3. The integrity of the occlusion balloon is checked by placing the distal end (balloon-tip) of the catheter into a basin of sterile saline solution while inflating the balloon with 20 c.c.s of air; if air bubbles are visualized leaking from balloon or balloon bond area replace cannula. The air should then be removed by gentle aspiration, completely collapsing the balloon against the main body of the arterial perfusion cannula. A 20 cc or syringe filled with normal saline solution should be used to prime the balloon and it's inflation channel. Remove all air from the balloon and inflation channel by aspiration of fluid from balloon and channel; after priming and removal of air close stopcock valve to balloon inflation channel leaving balloon collapsed around the main body of the arterial catheter. To avoid potential over inflation less than 35 ccs of solution should be reserved in the inflation syringe(s) for balloon inflation. The balloon catheter of the invention with the obturator inserted is placed to the side for later insertion.

If Fluoroscopic visualization of the cannula and balloon inflation is desired, a dilute intravenous contrast solution (10% CONRAY® or equivalent), diluted to a total of approximately 2% contrast, is prepared and used to prime the balloon and its inflation channel

4. The common femoral artery on the side selected for introduction of the cannula is surgically exposed, obtaining proximal and distal control of the vessel and any significant branches.

5. The patient is systemically anticoagulated as appropriate for cardiopulmonary bypass using heparin administered intravenously, with activated clotting times (ACT) determined in the routine fashion. A short vascular cannula with hollow-needle obturator is inserted into the femoral artery, with free blood return verifying intralumenal tip location. The needle obturator is removed, and a 0.035×180 cm stiff guide wire is introduced through the cannula and advanced cephalically up the aorta and across the aortic arch to position the tip in the ascending aorta; TEE imaging should be used to verify proper guide wire placement in the ascending aorta. Fluoroscopic visualization of the guide wire placement may also be used if desired.

6. During brief occlusion of the femoral artery the short femoral cannula is removed and a 1 cm transverse arteriotomy is created encompassing the site of the wire entry across the anterior arterial wall. The 0.035×180 cm Guide wire is back fed into the aortic root lumen of the arterial balloon catheter and through the hemostatic valve that comes attached to the lumen (see FIGS. 10A-10B for diagram of port, lumen and component locations and the previous discussion herein). Adjust the valve by tightening the thumbscrew of the hemostatic valve at port 116, tighten as much as possible while still allowing guide wire movement freely through the valve. The guide wire is left in position until the catheter insertion is completed. Use a soft-jaw clamp to control blood loss at femoral artery insertion site is recommended.

7. The arterial catheter is advanced over the guide wire into the femoral artery through the short sheath. The catheter (with obturator) is advanced in a retrograde fashion up the lilac artery, abdominal aorta and thoracic aorta. The arterial catheter is guided over the aortic arch with imaging assistance and the tip of the cannula is advanced into the ascending aorta. The position of the tip should be evaluated using TEE to verify that the tip is above and not interfering with the aortic valve. If Fluoroscopic visualization is desired; the radiopaque cannula marker at tip of the cannula can be used to assist placement. This will position the occlusion balloon in the ascending aorta, proximal to the origin of the innominate artery. In open sternotomy applications, tip position may be verified by direct palpation of the aortic root. The obturator is removed from the large central channel, which is de-aired by allowing back bleeding through ports 106 in FIG. 9, and then clamped at the ⅜ tubing connection 109 provided for clamping (see FIGS. 9, 10A and 10B for diagram of port, lumen and component locations). The obturator is appropriately set aside for reinsertion, if required.

8. The arterial perfusion lumen of the catheter is attached to the arterial blood supply line at 105 from the CPM, taking care not to introduce air at the site of connection (see FIGS. 9, 10A and 10B for diagram of port, lumen and component locations).

9. The inflation syringe filled with saline solution is attached via three-way valved manifold 119 (stopcock) to the occlusion balloon control lumen at 118. Pressure line from suitable pressure monitoring device should be attached to remaining valve port 120 to monitor balloon inflation pressure (see FIGS. 9, 10A and 10B for diagram of port, lumen and component locations).

10. The aortic root lumen labeled “k” is attached via three-way valved manifold 114 (stopcock) to the cardioplegia solution delivery/vent line from the CPM through 113. A pressure line from suitable pressure monitoring device should be attached to remaining valve port 114 to monitor cardioplegia or aortic root pressure. The cardiopulmonary bypass machine vent line is equipped with a ventricular vent valve to prevent excessive negative pressure on the vent line (see FIGS. 9, 10A and 10B for diagram of port, lumen and component locations).

11. Cardioplegic solution line pressure, aortic root pressure and balloon inflation pressure are measured at the appropriate ports as indicated (see FIGS. 9, 10A and 10B for diagram of port, lumen and component locations).

12. Venous cannulation is performed by direct cannulation of the right atrium with single or dual-stage cannula, selected cannulation of the superior and inferior vena cavas, or cannulation of the right atrium via the femoral, jugular or subclavian vein.

13. Cardiopulmonary bypass is initiated.

14. When aortic occlusion is required, the CPB blood flow is momentarily reduced to 25% and using the inflation syringe, the balloon is inflated to contact the vessel wall. After initial contact, under careful TEE monitoring (Fluoroscopic visualization of balloon inflation may also be used if desired), additional fluid should be added slowly until appropriate occlusion and stability are achieved. Inflation volume of 35 ccs or balloon pressure of 400 mmHg should not be exceeded. Full blood flow rate is then resumed. 10 ccs of volume will result in balloon diameter of 25-26 mm.

Inadequate venous drainage may allow the heart to eject against the balloon during inflation, resulting in balloon movement during inflation.

The right and left radial/brachial pressure waveforms are closely monitored during inflation, and the position of the balloon observed with TEE. Any change in the right radial/brachial waveform (in comparison to the left) may indicate that the occlusion balloon is obstructing the origin of the innominate artery, requiring deflation and repositioning.

The right and left arterial waveforms are monitored and evaluated continuously during the period of balloon inflation. Any change in the right radial/brachial waveform (in comparison to the left) may indicate that the occlusion balloon is obstructing the origin of the innominate artery, requiring deflation and repositioning.

15. Cardioplegic solution is administered through the aortic root lumen as required to provide arrest. Prior to the delivery of cardioplegia, the aortic vent is stopped for 1-2 minutes to allow accumulation of blood at the aortic root. The aortic root lumen is then cleared of air by gentle aspiration or gravity blood flow back through the lumen, then the cardioplegia solution can be administered through the lumen. The cardioplegia flow should begin slowly, and gradually be increased to the desired flow and pressure. The position of the occluding balloon should be closely observed for shifts during the delivery of cardioplegia, and verified again after cessation of the cardioplegia delivery.

16. The aortic root lumen may be opened to the CPB vent line when cardioplegia is not being administered. A safety valve should be inserted into the vent line to prevent more than 80 mmHg of vacuum. It is recommended that the surgical field be flooded with CO2 to prevent air introduction.

17. When aortic occlusion is no longer required gently aspirate fluid from balloon until total volume used for inflation is returned to syringe; close stopcock to balloon inflation lumen to assure balloon is collapsed against cannula. Cannula may now be withdrawn at the conclusion of bypass.

18. To remove catheter after conclusion of bypass, withdraw catheter to indicator mark indicating distal blood outlet port is two inches from arterial access incision, clamp cannula at indicator mark using tube-occluding forceps. A sterile towel should be wrapped around catheter covering exposed portion of catheter between indicator mark and distal end of catheter; this will control blood loss during catheter withdrawal. If obturator reinsertion is desired, obturator may now be inserted back into catheter up to position of clamp. Clamp should be removed and obturator advanced to incision site. Catheter can now be withdrawn on to obturator and access incision closed.

Should change out of the catheter be required during cardiopulmonary bypass:

1. Completely deflate occlusion balloon,

2. Insert 0.035×180-cm stiff guide wire through hemostatic valve attached to aortic root lumen, adjust valve to control bleed-back while still allowing free movement of guide wire. Use TEE and/or fluoroscopic imaging to position tip of guide wire in ascending aorta at tip of aortic cannula.

3. Prepare new arterial catheter for introduction as specified in directions for use item 3.

4. Discontinue arterial blood flow from cardiopulmonary bypass machine.

5. Clamp arterial cannula at ⅜ tubing section provided for clamping. Clamp cardiopulmonary bypass machine arterial line just distal of the arterial perfusion catheter connection. Separate connection between arterial perfusion catheter and cardiopulmonary bypass machine arterial line.

6. Withdraw arterial perfusion catheter over guide wire and remove arterial perfusion from guide wire taking care not to change position of guide wire in aorta. Use of a soft-jaw clamp to control blood loss at femoral artery insertion site is recommended.

7. Advance new arterial perfusion catheter over guide wire, balloon first into the femoral artery. The arterial perfusion catheter (with obturator) is advanced in a retrograde fashion up the lilac artery, abdominal aorta and thoracic aorta. When the arterial perfusion catheter has been dvanced past the black port indicator markers, the obturator can be removed from the catheter, which is de-aired by allowing back bleeding, and then clamped at the ⅜ tubing area provided for clamping. The cardiopulmonary bypass machine arterial line may now be connected to the catheter, taking care not to introduce any air into the line while connecting. Bypass may now be reinitiated.

The arterial perfusion catheter should then be positioned and used as referred to in directions for use items 7 through 18.

All publications and patent applications mentioned in this specification are herein incorporated by reference to the same extent as if each individual publication or patent application was specifically and individually indicated to be incorporated by reference.

The invention now being fully described, it will be apparent to one of ordinary skill in the art that many changes and modifications can be made thereto without departing from the spirit or scope of the appended claims.

What is claimed is:

1. A balloon catheter for delivering blood to an animal while blocking the aortic arch between the great arteries and the coronary ostia, the balloon catheter having a distal portion conjoined with a proximal portion, wherein:
(A) the distal portion comprises:
(a) an elongated, flexible shaft having distal and proximal ends and further having at least two lumens extending about the length of the shaft independent of and parallel to each other,
(b) the first lumen having an opening at both the distal and proximal ends of the shaft,
(c) an inflatable balloon integrated into the shaft near the distal end of the shaft,
(d) the second lumen having an opening at the proximal end of the shaft and an opening in fluid communication with the interior of the inflatable balloon, and
(e) the shaft having a non-traumatic distal tip and a length sufficient to traverse the aortic arch of a human;
(B) the proximal portion comprises a multi-lumen blood delivery portion having distal and proximal ends and being conjoined with the proximal end of the shaft at the distal end of the multi-lumen catheter, which multi-lumen blood delivery portion further comprises:
(a) a first lumen defined by a surrounding wall extending the length of the multi-lumen portion and being closed at its distal end but open at its proximal end for receiving extracorporeal blood from a cardiopulmonary machine,
(b) a second lumen (i) extending the length of the multi-lumen portion parallel to the first lumen but independent thereof and (ii) open at its distal end, and
(c) third lumen that (i) is independent of and parallel to the first and second lumens, (ii) extends the length of the three-lumen portion, and (iii) is open at the distal end of the third lumen, wherein a plurality of outlet ports extend along the wall at the distal region of the proximal portion, the ports being in fluid communication solely with the interior of the first lumen; and
(C) the proximal end of the distal portion is conjoined with the distal end of the proximal portion so that the first lumen of the distal portion is in fluid communication solely with the second lumen of the proximal portion and the second lumen of the distal portion is in fluid communication solely with the third lumen of the proximal portion.

2. The balloon catheter of claim 1, wherein the proximal portion includes only three lumens and the distal portion of the balloon catheter includes only two lumens.

3. The balloon catheter of claim 2 having a length of about 75 cm to about 120 cm.

4. The catheter of claim 2, wherein the durometer rating of the distal portion is between about 60A and 90A.

5. The catheter of claim 2, wherein the second and third lumens of the proximal portion are positioned about 180° opposite of each other.

6. The catheter of claim 2, wherein the shaft of the distal portion is non-kinking.

7. The catheter of claim 2, wherein the first lumen of the distal portion has a diameter greater than the diameter of the second lumen of the distal portion.

8. The catheter of claim 2, wherein the combined cross-sectional area of the two lumens of the distal portion accounts for no more than about 50% of the cross-sectional area of the shaft.

9. The catheter of claim 8, wherein the combined cross-sectional area of the two lumens of the distal portion accounts for no more than about 40% of the cross-sectional area of the shaft.

10. The catheter of claim 2, wherein the balloon when inflated takes a cylindrical shape.

11. The catheter of claim 2, wherein the cross-sectional diameter of the distal portion is about 14-16 French and the cross-sectional diameter of the proximal portion is about 20-22 French.

12. The balloon catheter of claim 2, wherein the cross-sectional area of the first lumen of the proximal portion comprises at least 70% of the total cross-sectional area of the proximal portion.

13. The balloon catheter of claim 2 in combination with a flexible shaft designed to slidingly and snugly fit into the length of the first lumen of the proximal portion and block the outlet ports.

14. The balloon catheter of claim 2, wherein the plurality of outlet ports communicating with the first lumen of the proximal portion have an outflow capacity that exceeds the capacity for the extracarporeal blood to flow into the proximal end of the first lumen.

15. A method of performing cardiovascular surgery on a patient having a need thereof, which method comprises:
(A) inserting a balloon catheter having a distal balloon into the patient through the patient's femoral artery to position the balloon catheter so that the balloon is positioned in the ascending aorta between the patient's coronary ostia and great arteries;
(B) expanding the balloon to substantially block fluid communication between the patient's heart and the aorta;
(C) providing cardioplegia through the balloon catheter to the patient's heart to slow the heart rate;
(D) circulating blood from a cardiopulmonary machine through the balloon catheter to the patient's aorta and connected arteries; and
(E) performing the cardiovascular surgery on the patient, wherein the balloon catheter comprises:
a distal portion conjoined with a proximal portion, wherein:
(1) the distal portion comprises:
(a) an elongated, flexible shaft having distal and proximal ends and further having at least two lumens extending about the length of the shaft independent of and parallel to each other,
(b) the first lumen having an opening at both the distal and proximal ends of the shaft,
(c) an inflatable balloon integrated into the shaft near the distal end of the shaft,
(d) the second lumen having an opening at the proximal end of the shaft and an opening in fluid communication with the interior of the inflatable balloon, and
(e) the shaft having a non-traumatic distal tip and a length sufficient to transverse the aortic arch of a human;
(2) the proximal portion comprises a multi-lumen blood delivery portion having distal and proximal ends and being conjoined with the proximal end of the shaft at the distal end of the multi-lumen catheter, which multi-lumen blood delivery portion further comprises:
(a) a first lumen defined by a surrounding wall extending the length of the multi-lumen portion and being closed at its distal end but open at its proximal end for receiving extracorporeal blood from the cardiopulmonary machine,
(b) a second lumen (i) extending the length of the multi-lumen portion parallel to the first lumen but independent thereof and (ii) open at its distal end, and
(c) a third lumen that (i) is independent of and parallel to the first and second lumens, (ii) extends the length of the three-lumen portion and (iii) is open at the distal end of the third lumen, wherein a plurality of outlet ports extend along the wall at the distal region of the proximal portion, the ports in fluid communication solely with the interior of the first lumen; and
(3) the proximal end of the distal portion is conjoined with the distal end of the proximal portion so that the first lumen of the distal portion is in fluid communication solely with the second lumen of the proximal portion and the second lumen of the distal portion is in fluid communication with the third lumen of the proximal portion.

16. The method of claim 15, wherein the proximal portion of the balloon catheter includes only three lumens and the distal portion of the balloon catheter includes only two lumens.

17. The method of claim 16, wherein the balloon catheter is about 75 cm to about 120 cm in length.

18. The method of claim 16, wherein the durometer rating of the distal portion of the balloon catheter is between about 60A and 90A.

19. The method of claim 16, wherein the second and third lumens of the proximal portion of the balloon catheter are positioned about 180° opposite of each other.

20. The method of claim 16, wherein the shaft of the distal portion of the balloon catheter is non-kinking.

21. The method of claim 16, wherein the first lumen of the distal portion of the balloon catheter has a diameter greater than the diameter of the second lumen of the distal portion.

22. The method of claim 16, wherein the combined cross-sectional area of the two lumens of the distal portion of the balloon catheter accounts for no more than about 50% of the cross-sectional area of the shaft.

23. The method of claim 22, wherein the combined cross-sectional area of the two lumens of the distal portion of the balloon catheter accounts for no more than about 40% of the cross-sectional area of the shaft.

24. The method of claim 16, wherein the balloon of the balloon catheter when inflated takes a cylindrical shape.

25. The method of claim 16, wherein the cross-sectional diameter of the distal portion of the balloon catheter is about 14-16 French and the cross-sectional diameter of the proximal portion is about 20-22 French.

26. The method of claim 16, wherein the cross-sectional area of the first lumen of the proximal portion of the balloon catheter comprises at least 70% of the total cross-sectional area of the proximal portion.

27. The method of claim 16 is the balloon catheter in combination with a flexible shaft designed to slidingly and snugly fit into the length of the first lumen of the proximal portion and block the outlet ports during insertion into the patient's femoral artery.

28. The method of claim 16, wherein the plurality of outlet ports communicating with the first lumen of the proximal portion of the balloon catheter have an outflow capacity that exceeds the capacity for the extracarporeal blood to flow into the proximal end of the first lumen.

29. A method for preparing a balloon catheter, which method comprises:
(A) preparing a distal portion of the catheter that comprises:
(1) an elongated, flexible shaft having distal and proximal ends and further having at least two lumens extending about the length of the shaft independent of and parallel to each other,
(2) the first lumen having an opening at both the distal and proximal ends of the shaft,
(3) an inflatable balloon integrated into the shaft near the distal end of the shaft,
(4) the second lumen having an opening at the proximal end of the shaft and an opening in fluid communication with the interior of the inflatable balloon, and
(5) the shaft having a non-traumatic distal tip and a length sufficient to traverse the aortic arch of a human;
(B) preparing a proximal portion of the catheter that comprises a multi-lumen blood delivery portion having distal and proximal ends and being suitable for conjoining with the proximal end of the shaft of (A) at the distal end of the multi-lumen catheter, which multi-lumen blood delivery portion further comprises:
(1) a first lumen defined by a surrounding wall extending the length of the multi-lumen portion and being closed at its distal end but open at its proximal end for receiving extracorporeal blood from a cardiopulmonary machine,
(2) a second lumen (i) extending the length of the multi-lumen portion parallel to the first lumen but independent thereof and (ii) open at its distal end, and
(3) third lumen that (i) is independent of and parallel to the first and second lumens, (ii) extends the length of the three-lumen portion and (iii) is open at the distal end of the third lumen, wherein a plurality of outlet ports extend along the wall of the first lumen at the distal portion of the proximal portion, the ports in fluid communication solely with the interior of the first lumen; and
(C) aligning the proximal end of the distal portion with the distal end of the proximal portion so that the first lumen of the distal portion aligns with the second lumen of the proximal portion and the second lumen of the distal portion aligns with the third lumen of the proximal portion; and
(D) permanently conjoining the distal and proximal portions together so that the lumens aligned in part (C) above are in fluid communication with the other.

30. The method of claim 29, wherein the proximal portion of the balloon catheter comprises only three lumens and the distal portion of the balloon catheter comprises only two lumens.

31. The method of claim 30, wherein the balloon catheter has a length of about 75 cm to about 120 cm.

32. The catheter of claim 30, wherein the durometer rating of the distal portion is between about 60A and 90A.

33. The method of claim 30, wherein the second and third lumens of the proximal portion of the balloon catheter are positioned about 180° opposite of each other.

34. The method of claim 30, wherein the shaft of the distal portion of the balloon catheter is non-kinking.

35. The method of claim 30, wherein the first lumen of the distal portion of the balloon catheter has a diameter greater than the diameter of the second lumen of the distal portion.

36. The method of claim 30, wherein the combined cross-sectional area of the two lumens of the distal portion of the balloon catheter accounts for no more than about 50% of the cross-sectional area of the shaft.

37. The method of claim 36, wherein the combined cross-sectional area of the two lumens of the distal portion of the balloon catheter accounts for no more than about 40% of the cross-sectional area of the shaft.

38. The method of claim 30, wherein the balloon of the balloon catheter when inflated takes a cylindrical shape.

39. The method of claim 30, wherein the cross-sectional diameter of the distal portion of the balloon catheter is about 14-16 French and the cross-sectional diameter of the proximal portion is about 20-22 French.

40. The method of claim 30, wherein the cross-sectional area of the first lumen of the proximal portion of the balloon catheter comprises at least 70% of the total cross-sectional area of the proximal portion.

41. The method of claim 30 wherein a flexible shaft designed to slidingly and snugly fit into the length of the first lumen of the proximal portion of the balloon catheter is included with the balloon catheter.

42. The method of claim 30, wherein the plurality of outlet ports communicating with the first lumen of the proximal portion of the balloon catheter have an outflow capacity that exceeds the capacity for the extracarporeal blood to flow into the proximal end of the first lumen.

43. A multi-lumen balloon catheter for attachment to a another multi-lumen blood delivery catheter, the first multi-lumen balloon catheter comprising:
an elongated, flexible shaft having distal and proximal ends and further having at least two lumens extending about the length of the shaft independent of and parallel to each other,
the first lumen having an opening at both the distal and proximal ends of the shaft,
an inflatable balloon integrated into the shaft near the distal end of the shaft,
a second lumen having an opening at the proximal end of the shaft and an opening in fluid communication with the interior of the inflatable balloon,
the distal tip of the shaft having a blunt, nontraumatic design, and
the shaft having a length sufficient to traverse the aortic arch of a human.

44. The catheter of claim 43, wherein the length is about 15 cm to about 30 cm.

45. The catheter of claim 43, wherein the durometer rating is between about 60A and 90A

46. The catheter of claim 43, which is a two-lumen catheter wherein the first and second lumens are positioned about 180° opposite of each other.

47. The catheter of claim 46, wherein the first lumen has a diameter greater than the diameter of the second lumen.

48. The catheter of claim 43, wherein the shaft is non-kinking.

49. The catheter of claim 43, wherein the combined cross-sectional area of the lumens accounts for no more than about 50% of the cross-sectional area of the shaft.

50. The catheter of claim 43, which is a two-lumen catheter wherein the combined cross-sectional area of the two lumens accounts for no more than about 40% of the cross-sectional area of the shaft.

51. The catheter of claim 43, wherein the balloon when inflated takes a cylindrical shape.

52. The catheter of claim 43, wherein the balloon expands to a size that is about 10 mm to about 50 mm in length and is sufficient to block the ascending aorta.

53. The catheter of claim 43, wherein the flexibility is such that it is sufficient to traverse the aortic arch, while following the natural curvature of the aortic arch, thus allowing the catheter to be positioned in the ascending aorta such that the balloon is properly aligned.

54. A first multi-lumen blood delivery catheter having distal and proximal ends and being suitable for conjoining with multi-lumen shaft at the distal end of the first multi-lumen catheter, wherein the other multi-lumen shaft has at least one less lumen than the first multi-lumen catheter, which first multi-lumen catheter comprises:
(a) a first lumen defined by a surrounding wall extending the length of the multi-lumen catheter and being closed at its distal end but open at its proximal end for receiving extracorporeal blood from a cardiopulmonary machine,
(b) a second lumen (i) extending the length of the multi-lumen catheter parallel to the first lumen but independent thereof and (ii) open at its distal end, and
(c) third lumen that (i) is independent of and parallel to the first and second lumens, (ii) extends the length of the multi-lumen catheter and (iii) is open at its distal end,
wherein a plurality of outlet ports extend along the wall at the distal portion of the three-lumen catheter, the ports in fluid communication solely with the interior of the first lumen.

55. The multi-lumen catheter of claim 54, wherein the cross-sectional area of the first lumen comprises at least 70% of the total cross-sectional area of the three-lumen catheter.

56. The multi-lumen catheter of claim 54 in combination with a flexible shaft designed to slidingly and snugly fit into the length of the first lumen and block the outlet ports.

57. The multi-lumen catheter of claim 54, wherein the plurality of outlet ports communicating with the first lumen have an outflow capacity that exceeds the capacity for the extracarporeal blood to flow into the proximal end of the first lumen.

58. The three-lumen catheter of claim 54, wherein the overall length of the three-lumen catheter is about 60 cm to about 90 cm.

59. A balloon catheter for delivering blood to an animal while blocking the aortic arch between the great arteries and the coronary ostia, the balloon catheter having a distal blood delivery section and proximal blood transport section, wherein:
(A) the proximal blood transport section having distal and proximal ends, which blood transport section further comprises:
(a) a first blood transport lumen defined by a surrounding wall extending the length of the blood transport section at its proximal end for receiving extracorporeal blood from a cardiopulmonary machine and being open at its distal end,
(b) a second lumen (i) extending the length of the blood transport section parallel to the first lumen but independent thereof and (ii) open at its distal end for delivery of cardioplegia solution to the heart near the aortic root, and
(c) third lumen that (i) is independent of and parallel to the first and second lumens, (ii) extends the length of the blood transport section, (iii) is open at its distal end, and (iv) communicates with the interior of an inflatable balloon integrated into the distal region of the blood transport section;
(B) the distal blood delivery section comprises an extension of the first lumen of the blood transport section, the extension (i) being of a length to traverse at least a portion of the aortic arch, (ii) being in fluid communication with the first blood transport lumen, and (iii) having a plurality of outlet ports for delivery of blood in an antegrade fashion to the aorta; and
(C) the proximal end of the distal blood delivery section is conjoined with the distal end of the proximal blood transport section so that the extension of the first lumen is in fluid communication solely with the blood transport lumen of the proximal portion.

60. The catheter of claim 59, wherein the second and third lumens of the proximal portion are positioned about 180° opposite of each other.

61. The catheter of claim 59, wherein the balloon when inflated takes a cylindrical shape.

62. The catheter of claim 59, wherein the cross-sectional diameter of the distal blood delivery section is about 14-16 French and the cross-sectional diameter of the proximal blood transport section is about 20-22 French.

63. The balloon catheter of claim 59, wherein the plurality of outlet ports of the distal blood delivery section communicating with the first lumen of the proximal blood transport section have an outflow capacity that exceeds the capacity for the extracorporeal blood to flow into the proximal end of the first lumen.

64. The balloon catheter of claim 59, wherein the longitudinal axis of proximal blood transport section is positioned at an angle of about 110° to about 120° relative to the longitudinal axis of blood delivery section.

65. A method of performing cardiovascular surgery on a patient having a need thereof, which method comprises:
(A) inserting the balloon catheter of claim 59 into the patient through the patient's aortic artery to position the balloon catheter so that the balloon is positioned in the ascending aorta between the patient's coronary ostia and great arteries and the blood delivery section is positioned to traverse a portion of the patient's aortic arch;
(B) inflating the balloon with a fluid transported through the third lumen to substantially block fluid communication between the patient's heart and the aorta;
(C) providing cardioplegia through the second lumen of the blood transport section to the patient's heart to slow the heart rate;
(D) circulating blood from a cardiopulmonary machine through the outlet ports of the blood delivery section of the first lumen to the patient's aorta and connected arteries; and
(E) performing the cardiovascular surgery on the patient.

65. The method of claim 65, wherein the second and third lumens of the proximal blood transport section of the balloon catheter are positioned about 180° opposite of each other.

67. The method of claim 65, wherein the balloon of the balloon catheter when inflated takes a cylindrical shape.

68. The method of claim 30, wherein the cross-sectional diameter of the distal blood delivery section of the balloon catheter is about 14-16 French and the cross-sectional diameter of the proximal blood transport section is about 20-22 French.

69. The method of claim 65, wherein the plurality of outlet ports communicating with the first lumen of the proximal blood delivery section of the balloon catheter have an outflow capacity that exceeds the capacity for the extracorporeal blood to flow into the proximal end of the first lumen.

70. The method of claim 65, wherein the longitudinal axis of the proximal blood transport section is positioned at an angle of about 110° to about 120° relative to the longitudinal axis of the distal blood delivery section.
